SEQUENCE LISTING (34 sequences)

SEQ ID NO: 1 - protein, 59 aa, Dendroaspis jamesonii kaimosae
  ref <302>: TITLE: Dendroaspin Molecules
  ref <309>: DATABASE ENTRY DATE:
  ref <310>: PATENT DOCUMENT NUMBER: WO 98/42834
  ref <311>: PATENT FILING DATE: 1998-03-20
  ref <312>: PUBLICATION DATE: 1998-10-01
RICYNHLGTKPPTTETCQEDSCYKNIWTFDNIIRRGCGCFTPRGDMPGPYCCESDKCNL

SEQ ID NO: 2 - protein, 59 aa, Artificial sequence
  misc_feature: Description of Artificial Sequence: Modified Dendroaspin (KGD-dendroaspin)
RICYNHLGTKPPTTETCQEDSCYKNIWTFDNIIRRGCGCFTPKGDMPGPYCCESDKCNL

SEQ ID NO: 3 - protein, 59 aa, Artificial sequence
  misc_feature: Description of Artificial Sequence: Modified Dendroaspin (KQA-dendroaspin)
RICYNHLGTKPPTTETCQEDSCYKNIWTFDNIIRRGCGCFTPKQAMPGPYCCESDKCNL

SEQ ID NO: 4 - protein, 59 aa, Artificial sequence
  misc_feature: Description of Artificial Sequence: Modified Dendroaspin (KGDW-dendroaspin)
RICYNHLGTKPPTTETCQEDSCYKNIWTFDNIIRRGCGCFTPKGDWPGPYCCESDKCNL

SEQ ID NO: 5 - protein, 59 aa, Artificial sequence
  misc_feature: Description of Artificial Sequence: Modified Dendroaspin (QGDM-dendroaspin)
RICYNHLGTKPPTTETCQEDSCYKNIWTFDNIIRRGCGCFTPQGDMPGPYCCESDKCNL

SEQ ID NO: 6 - protein, 58 aa, Artificial sequence
  misc_feature: Description of Artificial Sequence: Modified Dendroaspin (LDV-dendroaspin)
RICYNHLGTKPPTTETCQEDSCYKNIWTFDNIIRRGCGCFTPLDVPGPYCCESDKCNL

SEQ ID NO: 7 - protein, 59 aa, Aritifical sequence
  misc_feature: Description of Artificial Sequence: Modified Dendroaspin (AGDV-dendroaspin)
RICYNHLGTKPPTTETCQEDSCYKNIWTFDNIIRRGCGCFTPAGDVPGPYCCESDKCNL

SEQ ID NO: 8 - protein, 59 aa, Aritifical sequence
  misc_feature: Description of Artificial Sequence: Modified Dendroaspin (AGDM-dendroaspin)
RICYNHLGTKPPTTETCQEDSCYKNIWTFDNIIRRGCGCFTPAGDMPGPYCCESDKCNL

SEQ ID NO: 9 - protein, 61 aa, Artificial sequence
  misc_feature: Description of Artificial Sequence: Modified Dendroaspin (KQAGD-dendroaspin)
RICYNHLGTKPPTTETCQEDSCYKNIWTFDNIIRRGCGCFTPKQAGDVPGPYCCESDKCNL

SEQ ID NO: 10 - protein, 59 aa, Artificial sequence
  misc_feature: Description of Artificial Sequence: Modified Dendroaspin (QGDV-dendroaspin)
RICYNHLGTKPPTTETCQEDSCYKNIWTFDNIIRRGCGCFTPQGDVPGPYCCESDKCNL

SEQ ID NO: 11 - protein, 59 aa, Artificial sequence
  misc_feature: Description of Artificial Sequence: Modified Dendroaspin (DGEA-dendroaspin)
RICYNHLGTKPPTTETCQEDSCYKNIWTFDNIIRRGCGCFTPDGEAPGPYCCESDKCNL

SEQ ID NO: 12 - protein, 59 aa, Artificial sequence
  misc_feature: Description of Artificial Sequence: Modified Dendroaspin (DGPA-dendroaspin)
RICYNHLGTKPPTTETCQEDSCYKNIWTFDNIIRRGCGCFTPDGPAPGPYCCESDKCNL

SEQ ID NO: 13 - protein, 73 aa, Artificial sequence
  misc_feature: Description of Artificial Sequence: Modified Dendroaspin (KGDM-dendroaspin-Hr)
RICFTPRGDMPGPYPGPCQEDSCYKNIWTFDNIIRRGCGCFTPKGDMPGPYCFPRPQSHNDGDFEEIPEEYLQ

SEQ ID NO: 14 - protein, 70 aa, Artificial sequence
  misc_feature: Description of Artificial Sequence: Modified Dendroaspin (QGDV-dendroaspin-Tm)
RICFTPRGDMPGPYPGPCQEDSCYKNIWTFDNIIRRGCGCFTPQGDVPGPYECPEGYILDDGFICTDIDE

SEQ ID NO: 15 - protein, 66 aa, Artificial sequence
  misc_feature: Description of Artificial Sequence: Modified Dendroaspin (KGDW-dendroaspin-Gb)
RICFTPRGDMPGPYPGPCQEDSCYKNIWTFDNIIRRGCGCFTPKGDWPGPYGDTDLYDYYPEEDTE

SEQ ID NO: 16 - DNA, 84 bp, Artificial sequence
  misc_feature: Description of Artificial Sequence: Deoxyoligonucleotide sequence NP1
gggatccttgtcccgcgtaaagcttacccggaatgcggtgaaaacgaatggcttgacgactgcggtactcagaaaccgtgcgaa

SEQ ID NO: 17 - DNA, 87 bp, Artificial sequence
  misc_feature: Description of Artificial Sequence: Deoxyoligonucleotide sequence NP2
gcatttagcttcgcacggtttctgagtaccgcagtcgtcaagccattcgttttcaccgcattccgggtatgctttacgcgggacaag

SEQ ID NO: 18 - DNA, 87 bp, Artificial sequence
  misc_feature: Description of Artificial Sequence: Deoxyoligonucleotide sequence NP3
gctaaatgcaacgaagaaccgccggaagaagaagacccgatctgccgttctcgtggttgccttcttccgccggcttgcgtctgcaaa

SEQ ID NO: 19 - DNA, 90 bp, Artificial sequence
  misc_feature: Description of Artificial Sequence: Deoxyoligonucleotide sequence NP4
gtagaaaccgtctttgcagacgcaagccggcggaagaaggcaaccacgagaacggcagatcgggtcttcttcttccggcggttcttcgtt

SEQ ID NO: 20 - DNA, 82 bp, Artificial sequence
  misc_feature: Description of Artificial Sequence: Deoxyoligonucleotide sequence RN50
gacggtttctaccgtgacactgtcatcggtgactgcgtccgtgaagaagaatgcgaccagcatgaaatcatccatgtctgag

SEQ ID NO: 21 - DNA, 74 bp, Artificial sequence
  misc_feature: Description of Artificial Sequence: Deoxyoligonucleotide sequence RN60
aattctcagacatggatgatttcatgctggtcgcattcttcttcacggacgcagtcaccgatgacagtgtcacg

SEQ ID NO: 22 - DNA, 42 bp, Artificial sequence
  misc_feature: Description of Artificial Sequence: Deoxyoligonucleotide primer (xnp5)
gggatccatatcgaaggtcgtaaagcttacccggaatgcggt

SEQ ID NO: 23 - DNA, 54 bp, Artificial sequence
  misc_feature: Description of Artificial Sequence: Deoxyoligonucleotide primer (xnp3)
atgcccgggaccaccacccggaccaccaccaccgacatggatgatttcatgctg

SEQ ID NO: 24 - DNA, 30 bp, Artificial sequence
  misc_feature: Description of Artificial Sequence: Deoxyoligonucleotide primer (forward)
gggcccggggggcgtatctgctacaaccat

SEQ ID NO: 25 - DNA, 71 bp, Artificial sequence
  misc_feature: Description of Artificial Sequence: Deoxyoligonucleotide primer (reverse)
aattctcaaaggttgcatttgtcagattcgcagcagtacggacccggcatgtcaccacgagcagtgaagca

SEQ ID NO: 26 - protein, 149 aa, Artificial sequence
  Misc_feature: Description of Artificial Sequence: ND9
KAYPECGENEWLDDCGTQKPCEAKCNEEPPEEEDPICRSRGCLLLPACVCKDGFYRDTVIGDCVREEECDQHEIIHVGGGGPGPGGGPGGRICYNHLGTKPPTTETCQEDSCYKNIWTFDNIIRRGCGCFTARGDMPGPYCCESDNCNL

SEQ ID NO: 27 - protein, 13 aa, Artificial sequence
  Misc_feature: Description of Artificial Sequence: linker portion of ND9
GGGGPGPGGGPGG

SEQ ID NO: 28 - protein, 76 aa, Artificial sequence
  Misc_feature: Description of Artificial Sequence: NAP portion of ND9
KAYPECGENEWLDDCGTQKPCEAKCNEEPPEEEDPICRSRGCLLLPACVCKDGFYRDTVIGDCVREEECDQHEIIH

SEQ ID NO: 29 - protein, 156 aa, Artificial sequence
  misc_feature: Description of Artificial Sequence: protein fragment ND9-F1
GIHIEGRKAYPECGENEWLDDCGTQKPCEAKCNEEPPEEEDPICRSRGCLLLPACVCKDGFYRDTVIGDCVREEECDQHEIIHVGGGGPGPGGGPGGRICYNHLGTKPPTTETCQEDSCYKNIWTFDNIIRRGCGCFTARGDMPGPYCCESDNCNL

SEQ ID NO: 30 - protein, 112 aa, Artificial sequence
  misc_feature: Descriotion of Artificial Sequence: protein fragment ND9-F3
RSRGCLLLPACVCKDGFYRDTVIGDCVREEECDQHEIIHVGGGGPGPGGGPGGRICYNHLGTKPPTTETCQEDSCYKNIWTFDNIIRRGCGCFTARGDMPGPYCCESDNCNL

SEQ ID NO: 31 - DNA, 30 bp, Artificial sequence
  misc_feature: Description of Artificial Sequence: selection  primer
gaaggtcgtgggtaccatatcgaaggtcgt

SEQ ID NO: 32 - DNA, 36 bp, Artificial sequence
  misc_feature: Description of Artificial Sequence: mutagenesis primer
tgcttcactccgaaaggtgacatgccgggtccgtac

SEQ ID NO: 33 - DNA, 48 bp, Artificial sequence
  misc_feature: Description of Artificial Sequence: mutagenesis deoxyoligonucleotide
ggttgcttcactccgaaacaggctggtgacgttccgggtccgtactgc

SEQ ID NO: 34 - protein, 16 aa, Artificial sequence
  misc_feature: Description of Artificial Sequence: mutated peptide sequence
GCFTPKQAGDVPGPYC

(19) United States
(12) Patent Application Publication
(10) Pub. No.: US 2004/0072315 A1
(43) Pub. Date: Apr. 15, 2004

(54) Integrin-binding chimeras

(76) Inventors: Xinjie Lu, London (GB); Vijay Vir Kakkar, London (GB)

Correspondence Address: KLARQUIST SPARKMAN, LLP, 121 SW SALMON STREET, SUITE 1600, PORTLAND, OR, 97204, US

(21) Appl. No.: 10/467,020

(22) PCT Filed: Feb. 5, 2002
(86) PCT No.: PCT/GB02/00500

(51) Int. Cl. C12N 9/99

(52) U.S. Cl. 435/184; 525/54.1

(57) ABSTRACT

Products which contain two interlinked functional moieties of which one is an integrin-binding protein (e.g. a snake venom protein) or a homologue thereof. The products comprise a first portion which is an integrin-binding protein, a homologue thereof having a binding activity or a fragment of either which has integrin-binding activity, and, ligated to the first portion, a second portion which has a different function.

### FIELD OF THE INVENTION

The present invention relates to targeted drug therapy or bi- or multi-functional therapeutic compounds. More particularly it relates to chemical species which contain two interlinked functional moieties of which one is an integrin-binding protein (e.g. a snake venom protein) or a homologue thereof.

### BACKGROUND OF THE INVENTION

The role of blood coagulation is to provide an insoluble fibrin matrix for consolidation and stabilisation of a haemostatic plug (blood clot). Formation of a cross-linked fibrin clot results from a series of biochemical interactions involving a range of plasma proteins.

Acute vascular diseases, such as myocardial infarction, stroke, pulmonary embolism, deep vein thrombosis and peripheral arterial occlusion are caused by either partial or total occlusion of a blood vessel by a blood clot.

The formation of a blood clot within a blood vessel is termed thrombosis and is dependent upon platelet aggregation. In the context of blood vessel injury (such as that which might arise in surgical procedures), the interaction of blood platelets with the endothelial surface of injured blood vessels and with other platelets is a major factor in the course of development of clots or thrombi.

Platelet aggregation is dependent upon the binding of fibrinogen and other serum proteins to the glycoprotein receptor IIb/IIIa complex on the platelet plasma membrane. Glycoprotein GP IIb/IIIa is a member of a large family of cell adhesion receptors known as integrins, many of which are known to recognise an Arg-Gly-Asp (RGD) tripeptide recognition sequence.

Integrins are a family of cell surface receptors that mediate adhesion of cells to each other or to extracellular matrix substrate (1-5). They are composed of non-covalently associated α and β transmembrane subunits selected from among 16α and 8β subunits that heterodimerise to produce 20 receptors (6). Among the integrins, the platelet membrane α_{IIb}β₃ is the best characterised (3, 5). Upon cell activation, the α_{IIb}β₃ integrin (GP IIb/IIIa) binds several glycoproteins, predominantly through the Arg-Gly-Asp (RGD) tripeptide sequence (6-8) present in extracellular proteins such as fibrinogen (9), fibronectin (10), von Willebrand factor (11), vitronectin (12) and thrombospondin (13). The nature of the interactions between these glycoprotein ligands and their integrin receptors is known to be complex with conformation changes occurring in both the receptor (14) and the ligand (15).

The practical effect of such interactions can be illustrated by considering the treatment of localised narrowing of an artery caused by atherosclerosis. This is a condition which can usually be remedied surgically by the technique of balloon angioplasty. The procedure is invasive and causes some tissue damage to the arterial wall which can result in thrombus formation. Extracellular proteins such as fibronectin in the arterial wall become exposed to blood in the artery. Platelets bind to the RGD motif of fibronectin via integrin receptors which in turn leads to platelet aggregation and the start of the cascade of clotting reactions. An agent which specifically inhibits platelet aggregation at the sites of damage and which also inhibits clotting at these sites is required. The agent should be non-toxic and free of undesirable side effects such as a risk of generalised bleeding.

Various agents for preventing formation of blood clots are now available, such as aspirin, dipyridamole and filopidine. These products generally inhibit platelet activation and aggregation, or delay the process of blood coagulation, but they have the potential side effect of causing prolonged bleeding. Moreover, the effect of such products can be reversed only by new platelets being formed or provided.

Therefore, the development of antagonists towards selected cell adhesion events would be of significant clinical utility in the treatment of thrombosis and atherosclerosis. A key cell adhesion mechanism common to a number of integrin-ligand interactions involves the recognition of aspartic acid (D)-containing sequences or motifs identified by the use of inhibitory synthetic peptide analogues including RGD, KGD, LDV, KQAGDV. However, these peptides are limited by low potency and specificity. In this regard, a major breakthrough has been the discovery of a family of small, RGD-containing proteins derived from snake venoms termed disintegrins.

Scarborough et al (17) have reported a naturally occurring KGD-containing snake protein isolated from the venom of Sistrurus M. Barbouri termed barbourin showing a GPIIb-IIIa specific integrin antagonist activity.

Recently, many proteins from a variety of snake venoms have been identified as potent inhibitors of platelet aggregation and integrin dependent cell adhesion. The majority of these proteins which belong to the disintegrin family share a high level of sequence homology, are small (4-8 kDa), cysteine rich and contain the sequence RGD (16) or KGD (17). In addition to the disintegrin family, a number of non-disintegrin RGD proteins of similar inhibitory potency, high degree of disulphide bonding and small size, have been isolated from both the venoms of the Elapidae family of snakes (18, 19) and leech homogenates (20). All of these proteins are approximately 1000 times more potent inhibitors of the interactions of glycoprotein ligands with the integrin receptors than simple linear RGD peptides—a feature that is attributed to the optimally favourable conformation of the RGD motif held within the protein scaffold. The NMR structures of several inhibitors including kistrin (21-23), flavorldin (24), echistatin (25-28), albolabrin (29), decorsin (30) and dendroaspin (31, 32) have been reported and the only common structural feature elucidated so far is the positioning of the RGD motif at the end of a solvent exposed loop, a characteristic that is of prime importance to their inhibitory action.

Dendroaspin, therefore, is a natural variant of the short neurotoxin family, but contains the adhesive tripeptide Arg-Gly-Asp (RGD) and functions as a potent antagonist of integrin-mediated cell adhesive interactions. Dendroaspin was originally isolated from the venom of the Elapidae snake Dendroaspis jamesonil (Jameson's mamba) as a potent inhibitor of platelet aggregation and integrin mediated platelet adhesion. The activity of dendroaspin is due to an RGD motif contained within a solvent-exposed loop. International patent application WO 98/42834 describes amongst other things bi- or multi-functional molecules based on a dendroaspin scaffold, in which, in addition to integrin-binding function, a second function is achieved by adding a domain of another protein to a dendroaspin scaffold. WO 98/42834 and its entire content is included herein by reference, as is the disclosure of the corresponding U.S. application Ser. No. 09/381,546.

As is described in WO 98/42834, the dendroaspin molecule has 59 amino acid residues and comprises 3 loops. Loop I comprises amino residues 4-16, loop II residues 23-36 and loop III residues 40-50; it is loop III which contains the RGD motif in wild-type dendroaspin. The RGD domain forms residues 43-45.

As discussed above, therefore, the response to vascular injury results in the sequential formation of the serine proteases factor Xa and α-thrombin. The factor Xa functions at the intersection of the intrinsic and extrinsic pathway for blood coagulation, activates prothrombin to thrombin mediated by the catalytic prothrombinase complex composed of cofactor Va, and acidic phospholipid membranes in the presence of calcium ions. Thrombin is the principal mediator of the thrombotic response through its role as the primary agonist of platelet activation and subsequent aggregation and, through the proteolytic conversion of soluble fibrinogen to insoluble fibrin, both of which result in the formation of an intravascular thrombus. The central role of factor Xa in blood coagulation suggests that factor Xa inhibitors will have therapeutic utility as anticoagulants. Consequently, a number of factor Xa inhibitors such as tick and nematode anticoagulant peptides were originally isolated from Ornithodoros moubata (TAP) and hookworm Ancylostoma caninum (ACAP, NAP), respectively.

NAPs are discussed by Stanssens et al (39), Duggan et al (40) and Rebello et al (41), the disclosures of all of which are included herein by reference. Three different proteins (NAP5, NAP6 and NAPc2) with anticoagulant properties have been identified in the hookworm Ancylostoma caninum. These NAPs are highly potent and specific inhibitors of Factors VIIa and Xa. When tested against 11 other serine proteases, NAP5 showed 50% inhibition of just Factor Xa, while NAP6 and NAPc2 did not significantly inhibit any of the other proteases. NAP5 and NAP6 inhibit thrombin formation by direct binding to the catalytic site of Factor Xa; NAPc2 inhibits thrombin by binding to Factor Xa at an exo-site and by the binding of the resultant complex to the tissue factor-Factor VIIa complex. These NAP5 are 75-84 residues long and contain 10 cysteine residues paired into 5 disulfides (see FIG. 1 of Duggan for the sequences).

The preparation and characterisation of rNAP5 is disclosed by Stanssens et al.

Proteins having Factor Xa inhibitory activity and having one or more NAP domains, as well as the expression and use of such proteins, are described in U.S. Pat. No. 6,121,435, which is a continuation of co-pending application U.S. Ser. No. 08/809,455 filed on Apr. 17, 1997, which was a 371 of PCT/US95/13231, filed Apr. 17, 1997 and Continuation-In-Part of U.S. Ser. No. 08/461,965, now U.S. Pat. No. 5,872,098, Ser. No. 08/465,380, now U.S. Pat. No. 5,863,894, Ser. No. 08/486,397, now U.S. Pat. No. 5,866,542 and Ser. No. 08/486,399, now U.S. Pat. No. 5,866,543, all filed on Jun. 5, 1995, each of which is a continuation-in-part of U.S. Ser. No. 08/326,110, now U.S. Pat. No. 5,945,275, filed Oct. 15, 1994; the disclosures of all these patents are incorporated herein by reference.

TAP is a low molecular weight serine protease inhibitor. The peptide is a slow, tight-binding inhibitor, specific for factor Xa (Ki=0.588+/−0.054 nM). The inhibitor also acts as an anticoagulant in several human plasma clotting assays in vitro. TAP inhibits only factor Xa: it has been found to have no effect at a 300-fold molar excess on factor VIIa, kallikrein, trypsin, chymotrypsin, thrombin, urokinase, plasmin, tissue plasminogen activator, elastase, or Staphylococcus aureus V8 protease (Waxman et al, 42). The characteristics and expression of TAP are described also in U.S. Pat. No. 5,239,058; the disclosures of Waxman and U.S. Pat. No. 5,239,058 are included herein by reference.

Also as discussed above, therefore, it is well known that the binding of platelets to fibrinogen is an essential step in the formation of a platelet-rich blood clot and is mediated by the integrin α_{IIb}β₃ found on the surface of activated platelets. Upon cell activation, the α_{IIb}β₃ integrin binds several glycoproteins, predominantly through the Arg-Gly-Asp (RGD) tripeptide sequence present in extracellular matrix proteins. Potentially, an α_{IIb}β₃ integrin antagonist could be used as an antiplatelet aggregation agent for the treatment of thrombotic diseases. Snake venoms contain a variety of proteins, some of which contain an Arg-Gly-Asp (RGD) or Lys-Gly-Asp (KGD) sequence and can act as potent inhibitors of platelet aggregation and integrin mediated platelet adhesion. These molecules include the disintegrin family and dendroaspin.

### SUMMARY OF THE INVENTION

The following abbreviations are used in this specification:

Hydrophobic Amino Acids

A=Ala=alanine

V=Val=valine

I=Ile=isoleucine

L=Leu=leucine

M=Met=methionine

F=Phe=phenylalanine

P Pro=proline

W=Trp=tryptophan

Polar (Uncharged) Amino Acids

N=Asn=asparagine

C=Cys=cysteine

Q=Gin=glutamine

G=Gly=glycine

S=Ser=serine

T=Thr=threonine

Y=Tyr=tyrosine

Positively Charged Amino Acids

R=Arg=arginine

H=His=histidine.

K=Lys=lysine

Negatively Charged Amino Acids

D=Asp=aspartic acid

E=Glu=glutamic acid

So as to create potent inhibitors having bifunctional activity and specifically showing both anti-coagulation and anti-platelet activities, the inventors have developed an effective chimeric product composed of the interlinked functional domains of an integrin-binding protein and another functional species. A specific chimera in this case is of an anti-factor Xa NAP and an anti-platelet dendroaspin, and potentially inhibits both platelet aggregation and the serine protease factor Xa at nanomolar scales. This inhibitory action is surprisingly good compared to previous bi-functional anti-coagulation and anti-aggregation molecules.

The invention therefore provides a product having a first portion, which has a binding activity and is an integrin-binding protein (especially a snake venom protein), a homologue thereof or a fragment of either, ligated to a second portion which has a different function and, preferably, to another protein. The ligation may be direct but is preferably through a linker, particularly but not necessarily a peptide linker. The invention particularly provides products comprising a NAP-based domain linked to an integrin-binding domain, for example a domain containing RGD or KGD or a dendroaspin-based domain.

The linker, when present preferably comprises an imino acid, for example it may comprise the amino acid sequence PG or GP or homologues thereof. In which one or both P (proline) residues are replaced by another imino acid. Such linkers are new and are themselves included in the invention, as are poly(amino acid) molecules comprising two domains having different activities interlinked by a linker comprising an imino acid residue. The two domains may comprise one domain derived from a first native molecule and another domain foreign to the first native molecule, for example derived from a second native molecule; in one class of molecules, the molecules are not wild-type molecules.

### DETAILED DESCRIPTION OF THE INVENTION

The present invention therefore relates in one aspect to products comprising a first portion which is an integrin-binding protein, a homologue thereof having a binding activity or a fragment of either which has a binding activity, preferably integrin-binding activity, and, ligated to the first portion, a second portion which has a different function. The integrin-binding protein is most often a protein which binds to GP IIb/IIIa. RGD and KGD proteins are particularly preferred. The terms “first” and “second” here do not refer to the sequence of the two portions, since the invention includes products in which the first portion is linked to the C-terminus of the second portion (as is preferred in one class of product) or vice-versa.

Throughout the description and claims of this specification, the word “comprise” and variations of the word, such as “comprising” and “comprises”, means “including but not limited to,” and is not intended to exclude other components, integers, additives or steps. The protein is preferably a snake venom protein. In one preferred class of products, the protein is a disintegrin or a protein with a disintegrin-like domain (especially an RGD disintegrin-like domain), for example an MDC enzyme [MDC enzymes are large metalloproteinases composed of an N-terminal Metalloproteinase domain, a Disintegrin-like domain and a Cys-rich C-terminus). In contrast, disintegrins are small non-enzymatic RGD-containing cysteine-rich polypeptides]. Exemplary proteins are MDC-15 (metargidin), rhodostomin, accutin, applaggin, kistrin, flavoridin, batroxostatin, elegantin, jararacin, lachesin, basilicin, cereberin, viridin, molossin, echstatin, albolabrin, decorsin or dendroaspin.

The first portion may comprise a non-wild-type protein. In particular, it may be a homologue of a wild-type protein, modified at the integrin-binding domain to have another integrin-binding motif or to have another binding activity; additionally or alternatively the protein may have been modified elsewhere, for example to add a further functional domain, to modify the activity of a functional domain (especially the integrin-binding domain) or as an artefact of production, for example an artefact of production as described below in relation to the second portion. The homologue preferably has at least 50% amino acid sequence homology with the wild-type protein, preferably at least 65% homology, more preferably at least 75% homology and most preferably at least 85% amino acid sequence homology with the wild-type protein.

In preferred products, the wild-type protein comprises an integrin-binding sequence which is RGD or KGD and a preferred class of their homologues contain in place of the native integrin-binding sequence another integrin-binding sequence comprising a tripeptide sequence containing D or E adjacent to G. Another integrin-binding motif useful in the products of the invention is LDV.

The invention includes products in which the first portion comprises a fragment of a protein or polypeptide mentioned above. Thus the invention includes products in which the first portion comprises a dendroaspin sequence, that is a sequence comprised in the native dendroaspin molecule. Generally stated, therefore, the invention includes products in which the first portion comprises a functional sequence contained in, or derived from, a native sequence.

The function of the second portion in one preferred class is a serine protease inhibitor function. The invention includes a class of products in which the second portion is an inhibitor of a component of the haemostatic system, especially the coagulation system. Most preferably the second portion comprises a protein or a polypeptide, which may have a wild-type sequence or a modification of a wild-type sequence. In the latter case, the protein may have been modified in its functional domain or elsewhere; for example it may have been modified to add a further functional domain, to modify the activity of a functional domain or as an artefact of production. As examples of artefacts of production may be mentioned one or more accidental mutations and the residue of a linker sequence to an affinity purification protein. If the protein or polypeptide is a modification of a native sequence, it preferably has a degree of homology with its wild-type protein which is at least 50%, preferably at least 65%, more preferably at least 75% and most preferably at least 85%.

In a particularly preferred class of products, the second portion comprises a wild-type TAP protein, NAP protein (notably NAP5 or NAP6) or ACAP protein or a homologue thereof. Alternatively the second portion may comprise a fragment of one of these, the fragment having inhibitor function of the same type as the corresponding wild-type protein.

The invention includes products in which the second portion comprises a fragment of a protein or polypeptide mentioned above, as for example in the case of the product termed Nb9-F3 (SEQ ID NO:30). The invention includes products in which the second portion is or comprises a poly(amino acid) having Factor Xa inhibitory activity and having one or more NAP domains as described or claimed in U.S. Pat. Nos. 6,121,435, 5,872,098, 5,863,894, 5,866,542, 5,866,543, or 5,945,275. Generally stated, therefore, the invention includes products in which the second portion comprises a functional sequence contained in, or derived from, a native sequence.

In many products of the invention the second portion comprises an amino acid sequence comprised in the sequence of amino acid residues from residue 1 to residue 40 of SEQ ID no: 30 (ND9-F3) or a homologue thereof; for example, the second portion may consist of, or include, amino acid residues 1 to 40 of SEQ ID no:30 (ND9-F3) or a homologue thereof comprising 1, 2 or 3 amino acid modifications (i.e. substitution, insertion and/or deletion of 1, 2 or 3 amino acid residues).

The invention includes a class of product in which the second portion consists of, or includes, an amino acid sequence comprised in the sequence of amino acid residues from residue 8 to 84 of SEQ ID NO:29 (ND9-F1) or a homologue thereof; for example, the second portion may consist of, or include, amino acid residues 8 to 84 of SEQ ID NO:29 (ND9-F1) or a homologue thereof comprising 1, 2 or 3 amino acid modifications (i.e. substitution, insertion and/or deletion of 1, 2 or 3 amino acid residues). It will be understood that the homologues of the second portion may contain more than 3 amino acid modifications, for example 4, 5 or even up to 10 modifications or more.

Some very preferred products have a first portion which is a dendroaspin-based sequence. Accordingly in exemplary products the first portion comprises a dendroaspin scaffold (either wild-type or a homologue, modified by deletion or replacement of the RGD motif and/or elsewhere).

Thus the invention includes products comprising a dendroaspin scaffold and, in particular, a serine protease inhibitor domain ligated to the dendroaspin scaffold. This class of products includes those in which the native RGD motif of dendroaspin has been replaced by a replacement amino acid sequence, which optionally is (i) an amino acid sequence having no integrin-binding activity or (ii) an integrin-binding amino acid sequence and comprising a tripeptide sequence other than RGD containing D or E adjacent to G.

The ligation is preferably performed through a linker, especially a linker which comprises or is a poly(amino acid). Suitably, the poly(amino acid) contains from 5 to 20 amino acid residues. Advantageously the linker contains an imino acid residue, for example proline, since this provides a turn or bend in the linker which can give rise to a conformation in which the two portions of the product are positioned so as not to interfere with each other. Whilst not bound by theory, it is believed that this conformational behaviour of imino acids, for example proline, may be due to the nature of the imino residue which restricts the conformational freedom of adjacent residues. Thus, the proline residue, the only common imino acid in proteins, has a bulky pyrrolidine ring that is believed to restrict the conformational range of adjacent residues. The lack of a proton on the imino nitrogen blocks hydrogen bond formation required for α-helix and β-sheet secondary structure, and thus disrupts the propagation of neighbouring secondary structures through interactive site(s).

For the purpose of disrupting the propagation of secondary structures and thus maintaining a separation between the two linked portions of the product, the linker advantageously comprises at least two imino acid residues. Some products of the invention comprise linkers which contain from 2 to 5 imino acid residues.

A particularly preferred class of linkers comprise at least a pair of non-adjacent imino acid residues, for example separated by from 1 to 10 amino acid residues, e.g. 1, 2, 3, 4, 5 or 6 amino acid residues. Typically at least one of the separating residue(s) is glycine, desirably glycine adjacent a member of the pair of non-adjacent imino acid residues; more desirably, each imino acid of the pair is adjacent a glycine residue desirably located between it and the other member of the pair.

Preferred imino acid residues are of the formula

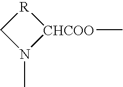

where R is —CH₂—, —CH₂—CH₂—, —S—CH₂—, or —CH₂—CH₂—CH₂—, which group is optionally substituted at one or more —CH₂— groups by from 1 to 3 C₁, C₂ or C₃ alkyl groups. In addition to prollne, other exemplary imino acids are 2- or 3-thiprolne and pipecolic acid.

Preferably, the products of the invention comprise a linker which contains an imino acid next to a glycine, i.e. which contains the sequence GP or PG or a homologue in which P is replace by another imino acid. More preferably, the or each imino acid residue of the linker is adjacent a glycine. (Glycine residues have no side chain, a feature which may avoid unwanted involvement of a side chain with the main chain). In some products, it is particularly preferred for at least one pair of adjacent imino acid and G residues to be in the sequence IA-G-IA, where each IA independently is an imino acid, notably proline.

One class of products comprises species in which the linker comprises a region, containing for example from 5 to 20 residues, consisting solely of glycine and imino acid residues. In preferred members of this class the region comprises the sequence IA-(G)ₙ-IA, preferably G-IA-G-IA-(G)ₙ-IA-G, where n is from 1 to 10 and preferably 2, 3, 4 or 5 (e.g. 3); an exemplary sequence is GGGG-IA-G-IA-GGG-IA-GG, in both these sequences, each IA independently is an imino acid, notably proline.

The invention therefore includes molecules comprising first and second portions interconnected by a linker. Typically, the second portion is a protein, as well as the first portion. In one class of molecules the linker consists of amino acids and the entire molecule may be a hybrid recombinant polypeptide.

The two portions of the product may be ligated together between positions of the two portions which facilitate or maximise the separation between the active domains of the two: for example, when the first portion is a dendroaspin, the second portion is preferably ligated to its N-terminal, relatively remote from loop III (which contains RGD in wild-type dendroaspin).

The linkers themselves are novel and form an aspect of the invention. Thus, the invention includes a linker comprising an amino acid sequence selected from the group consisting of Aa1-Gly and Gly-Aa1, wherein Aa1 is an imino acid. Preferably, the sequence is contained in the amino acid sequence Aa1-Gly-Aa2, where Aa1 and Aa2 are each independently an imino acid and preferably proline, as this sequence is believed to be particularly important for maintaining separation between the two linked domains, alternatively a plurality of Gly residues may be present between the two imino acid residues. More preferably, said sequence is contained in the amino acid sequence Gly-Aa1-Gly-Aa2-Gly.

It is common that both Aa1 and Aa2 are proline. Usually, but not always, the linker consists of a poly(amino acid) sequence containing from 5 to 20 amino acids. For example a suitable linker is shown in the sequences of ND9-F1 (SEQ ID NO:29) and ND9-F3 (SEQ ID NO:30) and comprises the sequence G G G G P G P G G G P G G (SEQ ID NO:27).

Preferred products of the invention are molecules comprising a dendroaspin scaffold as the first portion. The dendroaspin scaffold is usually linked through a proline-containing linker to a moiety which is typically a protein or polypeptide. The linker is preferably linked to the N-terminus of the dendroaspin scaffold. The moiety to which the dendroaspin scaffold is linked is preferably a serine protease inhibitor and typically selective for a chosen serine protease coagulation enzyme.

In the following detailed description, the invention is described by way of example with reference to dendroaspin scaffolds as the first portion of the product but it will be understood that the principles elucidated are applicable to other integrin-binding proteins and sequences. Thus, alternative integrin-binding protein scaffolds or their fragments may be used, whether in native or modified form.

The invention relates preferably to molecules comprising a dendroaspin (or other) scaffold ligated to a second portion which has a different function or, preferably, to another protein. The dendroaspin scaffold may have the wild-type sequence or be modified as compared with wild-type dendroaspin. The modification may comprise the deletion, addition or substitution of one or more amino acids and may merely be artefact(s) of the recombinant preparation of the molecule. Alternatively, the modification may be to change the properties of the dendroaspin scaffold, for instance loop III may be modified to change the binding properties of the RGD motif. In this respect, proline in loop III is believed to diverge the loop from the β-sheet. Thus proline residues, particularly either flanking the RGD sequence (P⁴²RGDMP⁴⁷) or neighbouring the RGD sequence (RGDMP⁴⁷GP⁴⁹), are believed to provide a favourable conformation binding to GPIIb/IIIa and it is preferred not to remove all of them when the dendroaspin is to have GPIIb/IIIa binding activity. In particular it is preferred not to remove both P⁴² and P⁴⁷, although it may be convenient to remove P⁴² (e.g. replace it with A), otherwise a thrombin cleavage site is formed.

Additionally or alternatively to modification of the flanking sequences, the dendroaspin (or other RGD protein) may be modified to replace the RGD motif with another integrin-binding motif. The invention includes molecules in which the native RGD motif has been replaced by an integrin-binding amino acid sequence other than RGD which contains aspartic acid (D) or glutamic acid (E), e.g. KGD. Preferably, the replacement integrin-binding amino acid sequence comprises a tripeptide sequence other than RGD containing D or E adjacent to G or to a hydrophobic amino acid. Alternatively, it may be LDV.

In some products the non-RGD tripeptide sequence is of the formula

B-J-Z

wherein

(I) J-Z is GD or GE and B is R, K, Q, A, H, N, A, V, I, L, M, F, P or W but is not R when J-Z is GD;

(II) B-J is DG or EG and Z is any amino acid; or

(III) I is D or E and B and Z are each independently selected from A, V, 1, L, M, F, P or W.

Preferably J-Z is GD and, in the products in which J-Z is GD or GE, B is preferably R, K, Q, A, H or N and more preferably is R, K, Q or A (but is not R when J-Z is GD).

A preferred class of products (I) comprises those in which B-J-Z is bonded at its C-terminal end to M, W, N or V. Preferably the M, W, N or V residue is followed by the P which is at position 47 of wild type dendroaspin or, less preferably, by an A residue substituted therefor.

Another preferred class of products (I) comprises those in which the integrin-binding amino acid sequence is preceded by the P which is at position 42 of wild type dendroaspin or, less preferably, by an A residue substituted therefor.

In some preferred products (I), especially those in which J-Z is GD, B is A, V, I, M, F, P, W and more preferably is L or V. The most preferred products of this type are those in which B is L and is preceded by M.

Preferred products (II) include those in which B-J is DG and/or Z is E, R or P, and especially in which Z is followed by the P which is at position 47 of wild type dendroaspin or by an A inserted before the wild type position 47 P.

A preferred class of products (III) comprises those in which J is D and, more particularly, B-J-Z is LDV. B-J-Z is preferably preceded by an I residue.

Of course, modified dendroaspins will often have a configuration which differs somewhat from that of wild-type dendroaspin but do normally have a three-loop structure. Preferably, any RGD-replacement associates with a receptor pocket or another pocket, since loop III is favourable for pocket-binding sequences; such sequences include a thrombin-binding sequence (GPRP is a thrombin-binding sequence) and the collagen α₂β₁-binding sequence DGE.

The invention includes products or hybrid polypeptides which comprise a modified dendroaspin scaffold which has an integrin-binding amino acid sequence at one domain and a non-dendroaspin amino acid sequence which confers a second functionality on another domain. The non-dendroaspin amino acid sequence may be comprised in the loops of the scaffold or be wholly or partially external to the loops. (The same applies to other loop-containing proteins). The dendroaspin-based products or polypeptides of the invention may therefore comprise in the dendroaspin scaffold at least one non-wild-type dendroaspin domain elsewhere than the native RGD site. The at least one non-wild-type dendroaspin domain usually comprises at least one non-dendroaspin sequence which confers functionality on the polypeptide. Such modified dendroaspin scaffolds are described in WO 98/42834 and in the US national phase application derived therefrom, U.S. Ser. No. 09/381,546, which is incorporated herein by reference.

One class of products have an integrin-binding activity which, when product molecules are administered in vivo, results in the binding of the molecules to platelets thereby inhibiting the aggregation of the platelets at sites of injury. In these products, the dendroaspin scaffold contains the RGD motif or another platelet-binding sequence, especially KGD. In addition to containing an integrin-binding domain, the dendroaspin scaffold may contain another non-wild-type dendroaspin domain which provides secondary, optionally further, functionality.

The dendroaspin scaffold is of course ligated to a species which has another functionality e.g. antithrombotic action, inhibition of cell migration and/or proliferation, or regulation of signal transduction. Such products are therefore bi- or multi-functional in their activities, and preferably are bi- or multi-functional in their activities against blood coagulation, particularly thrombus formation and arterial/venous wall thickening at the sites of injury. Products of the invention may have activity against leukocyte recruitment, immune system activation, tissue fibrosis or tumorigenesis. Those skilled in the art are familiar with peptide and peptidomimetic inhibitors of serine proteases (e.g. elastase, cathepsin G, urokinase (also called uPA), Factors II, IX, X, VII, IXa and XII thrombin, kallikrein, tissue plasminogen activator and plasmin) and the second portion may comprise such an inhibitor.

In particular the second portion is a protein or polypeptide, especially an inhibitor of a coagulation enzyme. Preferably, the second portion comprises wild-type TAP protein, NAP protein (notably NAP5) or ACAP protein or a homologue thereof having selective factor Xa inhibitory activity or, inhibitory activity selective for another serine protease, especially a coagulation serine protease.

The invention includes products in which the second portion confers platelet derived growth factor (PDGF) activity, glycoprotein IB activity, hirudin activity, thrombomodulin activity, vascular epidermal growth factor activity, transforming growth factor-1 activity, basic fibroblast growth factor activity, angiotensin II activity, factor VIII activity, tissue factor pathway inhibitor (TFPI) or von Willebrand factor activity. The second portion thus may comprise an amino add sequence derived from platelet derived growth factor (PDGF), glycoprotein IB, hirudin, thrombomodulin, vascular epidermal growth factor, transforming growth factor-1, basic fibroblast growth factor, angiotensin II, factor VIII, tissue factor pathway inhibitor (TFPI) or von Willebrand factor, or a functional sequence having homology to at least part of such sequence.

In this way the molecules of the invention may be rendered multifunctional so that they are active against, for example, platelet aggregation and another component in the dotting cascade (e.g. a serine protease coagulation enzyme, for example thrombin), or the intracellular signalling cascade (e.g. growth factor). The invention includes of course molecules which contain no integrin-binding function and molecules with no anti-coagulant function.

The products of the invention may comprise a dendroaspin scaffold having an amino acid sequence as shown in FIG. 1. Excluding the second portion and any linker, the products of the invention include polypeptides comprising dendroaspin scaffolds homologous to wild-type dendroaspin which may share about 50% or more amino acid sequence homology, preferably about 65% or more, more preferably about 75% or more and even more preferably about 85% or more homology with dendroaspin.

The dendroaspin scaffolds may comprise a greater or lesser number of amino acid residues compared to the 59 amino acids of dendroaspin. For example, the dendroaspin scaffolds may comprise a number of amino acid residues in the range 45 to 159, preferably about 49 to 89, more preferably about 53 to 69, even more preferably about 57 to 61. However, the invention includes polypeptides in which the dendroaspin scaffold contain 59 amino acid residues.

The invention includes species in which a foreign sequence is contained wholly within the dendroaspin scaffold (i.e. between residues 1 and 59, inclusive) and is for example loop grafted. Thus, in one class of products, a non-wild-type domain(s) is/are incorporated into (a) loop I and/or loop II; (b) loop I and/or loop III; (c) loop II and/or loop III; or loop I, loop II and loop III of the dendroaspin scaffold. If the polypeptide comprises a non-wild-type domain incorporated into a loop, the non-wild-type domain is in some polypeptides incorporated into either loop I or loop II, leaving loop III unaltered.

Another class, however, comprises dendroaspin scaffolds which comprise a non-wild-type domain extending into or substituting regions external to the loops, i.e. residues 1-3, 17-22, 37-39 and 51-59 such that residues of the non-loop regions are augmented or substituted for those of a further amino acid sequence being inserted (the non-wild-type domain).

The invention includes species in which a foreign sequence is contained within the scaffold of another integrin-binding protein.

Any inserted non-wild-type domain is preferably an amino acid sequence having no more than 100 amino acid residues, e.g. from 3 to 40 amino acid residues. Especially in the case of inserted sequences which are contained wholly within the dendroaspin scaffold, the non-wild-type domain more preferably has from 3-16, even more preferably 3-14 amino acid residues. When two non-wild-type domains are incorporated into the dendroaspin scaffold then the linear distance between these is preferably from 1-35 amino acids, more preferably 1-14 amino acids. When more than two non-wild-type domains are incorporated then there is preferably at least one native dendroaspin amino acid residue separating each further amino acid sequence.

Loop III of dendroaspin may be modified by insertion, deletion or substitution of any one or more amino acid residues, preferably a maximum of 8 or a minimum of 1 amino acids can be modified within loop III of dendroaspin, e.g. 1, 2, 3 or 4.

An integrin-binding sequence (e.g. KGD or RGD motif) may be incorporated into the dendroaspin scaffold or other integrin-binding polypeptide at a place other than the wild-type integrin-binding domain, preferably into loop I or loop II in the case of dendroaspin.

The dendroaspin-based molecules of the invention may comprise a dendroaspin loop III having an amino acid sequence flanking the RGD site modified from that flanking RGD in wild-type dendroaspin, for example modified as shown in FIG. 3B of WO 98/42834. An advantage of modifying the flanking region is that the activity (e.g. integrin-binding activity) of the sequence at the RGD site may be enhanced or become more specific for certain glycoprotein ligands. Also, if a modification of the dendroaspin scaffold has steric effects on a replacement amino acid sequence for RGD then loop III around the RGD domain can be modified to overcome any steric hindrance thereby at least partially restoring, perhaps enhancing, functionality at the RGD domain.

Especially if the RGD motif is replaced by an amino acid sequence having more than 3 residues, amino acids flanking the RGD site may be deleted, for example so that the number of amino acid residues in loop III remains as 13.

As indicated, modification of the dendroaspin loops may become necessary if a “foreign” further amino acid sequence incorporated into the dendroaspin scaffold has a steric hindrance effect either on another incorporated domain or on the loop III. Computer assisted molecular modelling using insight II software (Molecular Simulations Inc) can be used to predict the structure of the “loop grafted” dendroaspins scaffolds. In instances where steric effects between the loops may serve to cause loss of functionality, these effects can be “designed out” by modifying appropriate parts of the dendroaspin molecule in an appropriate way. Sometimes this may involve incorporating a number of suitable amino acid residues to extend one or more of the loop structures.

In the design of a bi-functional or multi-functional composition of matter in accordance with the invention, “fine tuning” of activity, stability or other desired biological or biochemical characteristic may be achieved by altering individual selected amino acid residues by way of substitution or deletion. Modification by an insertion of an amino acid residue or residues at a selected location is also within the scope of this “fine tuning” aspect of the invention. The site-directed mutagenesis techniques available for altering an amino acid sequence at a particular site in the molecule will be well known to a person skilled in the art.

The invention includes molecules comprising two domains, not both derived from the same native molecule, interlinked by a linker comprising an imino acid residue, as previously described. One of these domains may have platelet-binding, or GPIIb/IIIa-binding activity, and the other may be a second portion as described above in relation to the preferred products having a dendroaspin-based first portion.

The compounds of the invention may exist in different forms, such as acids, esters, salts and tautomers, for example, and the invention includes all variant forms of the compounds. In particular, the compounds may be in the form of acid addition salts which, for those compounds for pharmaceutical use, will be pharmaceutically acceptable. Exemplary acids include HBr, HCl and HSO₂CH₃.

The invention includes prodrugs for the active pharmaceutical species of the invention, for example in which one or more functional groups are protected or derivatised but can be converted in vivo to the functional group, as in the case of protected nitrogens. The term “prodrug,” as used herein, represents compounds which are rapidly transformed in vivo to the parent compound, for example, by hydrolysis in blood. A thorough discussion is provided in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins, et al. Synthetic Communications, 26(23), 4351-4367 (1996), each of which is incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, even if a protected derivative of compounds of the invention does not possess pharmacological activity as such, it may be administered, for example parenterally or orally, and thereafter metabolised in the body to form compounds of the invention which are pharmacologically active. Such derivatives are therefore examples of “prodrugs”. All prodrugs of the described compounds are included within the scope of the invention.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol; isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The invention therefore includes all variant forms of the defined compounds, for example any pharmaceutically acceptable salt, ester, acid or other variant of the defined compounds and their tautomers as well as substances which, upon administration, are capable of providing directly or indirectly a compound as defined above or providing a species which is capable of existing in equilibrium with such a compound.

Preparation

The products of the invention may comprise, and preferably are, polypeptides which may be made by construction of appropriate expression vectors, e.g. polynucleotides comprising a coding sequence operatively linked to a promoter. In other embodiments, the products of the invention are wholly or partly chemically synthesised.

In the case of products made in whole or in part using chemical synthesis, for example to ligate the two portions of the product together through a linker conventional technology may be used. Solid phase techniques may be used in whole or in part. The skilled reader will require no instruction in the chemical synthesis of peptides but ample instructions are to be found in “The Chemical Synthesis of Peptides”, John Jones, Clarendon Press, Oxford, England, 1991; Sharma, R. P.; Jones, D. A.; Broadbridge, R. J.; Corina, D. L. and Akhtar, M. A Novel Method of Solid Phase Synthesis Of Peptide Analogues, in Innovation and Perspectives in Solid Phase Synthesis, ed., R. Epton, 1994, Mayflower Worldwide Limited, Birmingham, page 353-356; Letsinger, R. L. and Kornet, M. J. J.Amer.Chem.Soc., 1963, 85, 3045.

A preferred technique for synthesis of peptides is solid phase synthesis, manual or automated, as first developed by Merrifield and described by Stewart et al. In Solid Phase Peptide Synthesis (1984). Chemical synthesis joins the amino acids in the predetermined sequence starting at the C-terminus. Basic solid phase methods require coupling the C-terminal protected.alpha.-amino acid to a suitable insoluble resin support. Amino acids for synthesis require protection on the α-amino group to ensure proper peptide bond formation with the preceding residue (or resin support). Following completion of the condensation reaction at the carboxyl end, the α-amino protecting group is removed to allow the addition of the next residue. Several classes of a-protecting groups have been described, see Stewart et al. in Solid Phase Peptide Synthesis (1984), with the acid labile, urethan-based tertiary-butyloxycarbonyl (Boc) being the historically preferred. Other protecting groups, and the related chemical strategies, may be used, including the base labile 9-fluorenylmethyloxycarbonyl (FMOC). Also, the reactive amino acid sidechain functional groups require blocking until the synthesis is completed. The complex array of functional blocking groups, along with strategies and limitations to their use, have been reviewed by Bodansky in Peptide Synthesis (1976) and, Stewart et al. in Solid Phase Peptide Synthesis (1984).

Solid phase synthesis is initiated by the coupling of the C-terminal α-protected amino acid residue. Coupling requires activating agents, such as dicyclohexycarbodiimide (DCC) with or without 1-hydroxybenzo-triazole (HOBT), diisopropylcarbodiimide (DIIPC), or ethyidimethylaminopropylcarbodiimide (EDC). After coupling the C-terminal residue, the α-amino protected group is removed by trifluoroacetic acid (25% or greater) in dichloromethane in the case of add labile tertiary-butyloxycarbonyl (Boc) groups. A neutralizing step with triethylamine (10%) in dichloromethane recovers the free amine (versus the salt). After the C-terminal residue is added to the resin, the cycle of deprotection, neutralization and coupling, with intermediate wash steps, is repeated in order to extend the protected peptide chain. Each protected amino acid is introduced in excess (three to five fold) with equimolar amounts of coupling reagent in suitable solvent. Finally, after the completely blocked peptide is assembled on the resin support, reagents are applied to cleave the peptide form the resin and to remove the side chain blocking groups. Anhydrous hydrogen fluoride (HF) cleaves the acid labile tertiary-butyloxycarbonyl (Boc) chemistry groups. Several nucleophilic scavengers, such as dimethylsulfide and anisole, are included to avoid side reactions especially on side chain functional groups.

Bispecific molecules of the present invention can be prepared by chemically conjugating the two portions (which may previously have been cloned or chemically synthesised) using methods well-known in the art. For example, a variety of coupling or cross-linking agents can be used for covalent conjugation. Examples of cross-linking agents include protein A, carbodjimide, N-succinimidyl-S-acetyl-thioacetate (SATA), N-succinimidyl-3-(2-pyridyidithio)-propionate (SPDP), and sulfosuccinimidyl 4-(N-maleimidomethyl) cyclohexane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described by Paulus (Behring Ins. Mitt. (1985) No. 78, 118-132); Brennan et al. (Science (1985) 229:81-83), and Glennie et al. (J. Immunol. (1987) 139: 2367-2375).

However, one preferred class of products comprises hybrid recombinant polypeptides. The skilled person can readily construct a variety of clones containing functional nucleic acids. Cloning methodologies to accomplish these ends, and sequencing methods to verify the sequences of nucleic acids, are well known in the art. Examples of appropriate cloning and sequencing techniques, and instructions sufficient to direct persons of skill through many cloning exercises are found in Sambrook, et al., Molecular Cloning: A Laboratory Manual (2nd Ed., Vols. 1-3, Cold Spring Harbor Laboratory (1989)), Methods in Enzymology, Vol. 152: Guide to Molecular Cloning Techniques (Berger and Kimmel (eds.), San Diego: Academic Press, Inc. (1987)), or Current Protocols in Molecular Biology, (Ausubel, et al. (eds.), Greene Publishing and Wiley-Interscience, New York (1987).

Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie A G, Buchs, Switzerland), invitrogen, San Diego, Calif., and Applied Biosystems (Foster City, Calif.), as well as many other commercial sources known to one of skill.

Polynucleotides containing a desired gene can be prepared by any suitable method including, for example, cloning and restriction of appropriate sequences as discussed supra, or by direct chemical synthesis by methods such as the phosphotriester method of Narang et al. Meth. Enzymol. 68: 90-99 (1979); the phosphodiester method of Brown et al., Meth. Enzymol. 68: 109-151 (1979); the diethylphosphoramidite method of Beaucage et al., Tetra. Lett., 22: 1859-1862 (1981); the solid phase phosphoramidite triester method described by Beaucage and Caruthers (1981), Tetrahedron Letts., 22(20):1859-1862, e.g., using an automated synthesizer, e.g., as described in Needham-VanDevanter et al. (1984) Nucleic Acids Res., 12:6159-6168; and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that, while chemical synthesis of DNA is limited to sequences of about 100 bases, longer sequences may be obtained by the ligation of shorter sequences.

Nucleic acids may be modified by site-directed mutagenesis, as is well known in the art. Native and other nucleic acids can be amplified by in vitro methods. Amplification methods include the polymerase chain reaction (PCR), the ligase chain reaction (LCR), the transcription-based amplification system (TAS), the self-sustained sequence replication system (SSR). A wide variety of cloning methods, host cells, and in vitro amplification methodologies are well-known to persons of skill.

The expression of rhodostomin is described in Chang H H, Hu S T, Huang T F, Chen S H, Lee Wu Y H, Lo S J. (1993). Rhodostomin, an RGD-containing peptide expressed from a synthetic gene in Escherichia coli, facilitates the attachment of human hepatoma cells, Biochem. Biophys. Res. Commun. 190:242-249, whilst the cloning and expression of recombinant kistrins is described by Tselepis V H, Green L J, Humphries M J, J Biol Chem Aug. 22, 1997;272(34):21341-8. Recombinant elegantins have been expressed by Salman Rahman, Alex Aitken, Geraldine Flynn, Caroline Formstone, Geoffrey F. Savidge, Biochem J Issue 2 (1998) pp 247-257.

In the case of dendroaspin-containing products and as described in WO 98/42834, the wild type dendroaspin gene may be successfully inserted into a plasmid pGEX-3×(FIG. 2 of WO 98/42834) and expressed according to the method of Lu et al, (1996) J Biol Chem 271: 289-295. Starting with the wild type gene for dendroaspin, variants of the dendroaspin gene for expressing polypeptides of the invention may then be engineered using recombinant DNA technology. For the longer insertion variants, oligonucleotides which encode the non-dendroaspin or heterologous amino acids may simply be inserted directly into suitably restriction digested wild type dendroaspin gene and then ligated. For minor changes such as modification of a few amino acid residues including the insertion, substitution or deletion, site directed mutagenesis may be used, for example using the Transformer™ Site-Directed Mutagenesis kit from Clontech Laboratories in accordance with the manufacturer's instructions.

As an alternative to modifying the wild type gene after insertion into an expression vector, as described above with reference to plasmid pGEX-3×, genes encoding polypeptides of the invention may be made by methods which comprise the construction of vectors containing non-wild-type genes by ligation of oligonucleotides optionally followed by modification by, in particular, site-directed mutagenesis.

FIG. 2A of WO 98/42834 shows the nucleotide sequence of a synthetic dendroaspin (Den) gene. The gene was designed on the basis of the known amino acid sequence (Williams J A et al ((1992)) Biochem Soc Trans 21: 735) and the codons for each amino acid were adopted from those which were highly expressed in E coli (Fiers W ((1982)) Gene 18: 199-209). Ten synthetic oligonucleotides are shown in brackets and numbered individually 1 to 10 either above the coding strand or below the non-coding strand. The stop codon is indicated by an asterisk. Three-letter amino acid code is used and the total of 59 amino acids of Den are only numbered 1 for N-terminal residue arginine and 59 for C-terminal leucine.

In an additional aspect, therefore, the invention resides in nucleic acid molecules encoding a polypeptide of the invention. The nucleic acid may be operatively linked to a promoter and optionally to a nucleic acid sequence encoding a heterologous protein or peptide thereby to encode a fusion product. Suitably the promoter is IPTG inducible and optionally the heterologous protein or peptide is glutathione 5-transferase.

Excluding the nucleic acid sequence encoding the second portion and any linker, nucleic acid sequences encoding the polypeptides of the invention may share about 50% nudeotide sequence homology, preferably about 65%, more preferably about 75% and even more preferably about 85% homology with a wild-type integrin-binding protein nucleotide sequence.

The invention includes plasmids comprising a nucleic acid of the invention, for example plasmid pGEX-3× comprising a nucleic acid of the invention, as well as host cells transformed with such a plasmid. A suitable host cell is E coli. The host cells may be provided as cell cultures.

Another aspect of the invention resides in a method of producing a polypeptide comprising culturing a host cell of the invention so as to express said polypeptide, extracting the polypeptide from the culture and purifying it.

The invention further includes a method of producing a polypeptide comprising an integrin-binding protein or its homologue, the method comprising:

a) preparing an expression vector comprising a nucleic acid sequence encoding a polypeptide product of the invention operatively linked to a promoter and optionally linked to a nucleic acid sequence encoding a heterologous protein for co-expression therewith; and

b) transforming a host cell with the vector and causing the host cell to express the nucleic acid sequence.

Some of these methods comprise

(i) assembling from overlapping oligonucleotides the coding sequence of an integrin-binding protein or a homologue thereof having a binding activity;

(ii) assembling from overlapping oligonucleotides the coding sequence of the second portion;

(iii) amplifying the coding sequences, the PCR primers being so designed to allow cloning of the integrin-binding protein and the second portion into an expression vector, the PCR primers optionally encoding a linker to interlink the integrin-binding protein and the second portion;

(iv) preparing an expression vector comprising the coding sequences operatively linked to a promoter and optionally linked to a nucleic acid sequence encoding a heterologous affinity purification protein for co-expression therewith.

In such methods, step (a) (ii) may comprise modifying the nucleic acid sequence of the vector by one or more of the insertion, deletion or substitution of nucleic acid residues.

Others of the methods comprise constructing from oligonucleotides an expression vector comprising a nucleic acid sequence encoding a dendroaspin (or other RGD protein) sequence in which the RGD-encoding domain has been deleted or replaced by a replacement amino acid sequence as defined herein and, optionally, modifying at least one other domain of the nucleic acid sequence of the vector encoding the dendroaspin scaffold by one or more of insertion, deletion or substitution of nucleic acid residues so that on expression the dendroaspin scaffold comprises a corresponding domain having a non-wild-type dendroaspin sequence.

The method may comprise the steps of:

a) extracting the expressed polypeptide from a host cell culture,

b) purifying the expressed polypeptide from the cell culture extract, and, if the expressed polypeptide is a fusion protein with a heterologous affinity purification protein, cleaving the desired product from the heterologous affinity purification portion of the fusion protein.

The heterologous affinity purification protein is suitably glutathione S-transferase (GST) and the purification suitably involves GST affinity chromatography followed by cleavage of the modified dendroaspin from GST.

Some products of the invention are made by producing an RGD-free dendroaspin as described above and chemically ligating a non-dendroaspin species to it.

Use

The peptides of the invention may be used for scientific investigations or as pharmaceuticals.

We have found that an integrin-binding protein provides an excellent scaffold for carrying “foreign” species, especially proteins, to potential targets. In this respect, the small size and conformational stability of the dendroaspin scaffold make it a particularly good model for pharmaceutical use. The products of the invention advantageously contain a platelet-binding domain in the first portion (e.g. dendroaspin) and have a second portion (e.g. a NAP or TAP) which inhibits a coagulation enzyme, preferably selectively. In this way, anti-coagulant activity can be targeted at areas where there are activated platelets and clot formation is commencing, potentially enabling beneficial localised anti-clotting activity without substantial systemic anti-clotting activity which can result in haemorrhaging. Alternatively, products may be prepared with other therapeutically useful bi- or multi-functional activities.

The pharmacologically active polypeptides may be formulated as a pharmaceutical composition comprising a polypeptide as hereinbefore defined, optionally further comprising a pharmaceutically acceptable excipient or carrier. A plurality of therapeutic (e.g. prophylactic) polypeptides of the invention of different functionalities may be combined together in a pharmaceutically acceptable form so as to provide a desired treatment, and/or they may be combined with one or more other therapeutic agents.

The compounds of the invention will normally be administered orally, intravenously, subcutaneously, buccally, rectally, dermally, nasally, tracheally, bronchially, by any other parenteral route, as an oral or nasal spray or via inhalation, More typically, the compounds will be administered by injection or infusion, especially intravenously. The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

The coagulation enzyme inhibitors of the invention may also be combined and/or co-administered with any antithrombotic agent with a different mechanism of action, such as the antiplatelet agents acetylsalicylic acid, ticlopidine, clopidogrel, thromboxane receptor and/or synthetase inhibitors, fibrinogen receptor antagonists, prostacyclin mimetics and phosphodiesterase inhibitors and ADP-receptor (P₂ T) antagonists.

The coagulation enzyme inhibitors of the invention may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

Envisaged suitable daily doses of the compounds of the invention in therapeutic treatment of humans are about 0.001-100 mg/kg body weight at peroral administration and 0.001-50 mg/kg body weight at parenteral administration. A preferred peroral dose of from 0.02 to 15 mg/Kg of body weight is envisaged, and the active compound may be given as a single dose, in multiple doses or as a sustained release formulation. For use with whole blood, from 1 to 10 mg per litre may be provided to prevent coagulation.

According to a further aspect of the invention there is thus provided a pharmaceutical composition including a compound of the invention, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminium monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed-absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic add; b) binders such as carboxymethylcellulose, alginates, gelatin, polyvinylpyrrolidone, sucrose and acada; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions which can be used include polymeric substances and waxes.

The active compounds may also be in micro-encapsulated form, if appropriate, with one or more of the above-mentioned exciplents.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form, can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Advantageously, the compounds of the invention are orally active, have rapid onset of activity and low toxicity.

One formulation may comprise extravasated blood combined with a polypeptide of the invention at a concentration in the range 1 nM-60 μM. This blood may be stored in ready to use form and provides an immediate and convenient supply of blood for transfusion in cases when clotting must be avoided such as during or immediately following surgical procedures.

The invention includes a therapeutic polypeptide as hereinbefore defined for use in medicine, preferably as a pharmaceutical.

The invention also provides for the use of a pharmacologically active polypeptide as hereinbefore defined for the manufacture of a medicament, which may for example be for the treatment or prophylaxis of disease associated with binding at a receptor or with thrombosis; more particularly thrombosis, myocardial infarction, retinal neovascularization, endothelial injury, dysregulated apoptosis, abnormal cell migration, leukocyte recruitment, immune system activation, tissue fibrosis and tumorigenesis.

The invention also provides methods for the treatment by therapy or prophylaxis of diseases associated with binding at a receptor or with thrombosis; more particularly thrombosis, myocardial infarction, retinal neovascularization, endothelial injury, dysregulated apoptosis, abnormal cell migration, leukocyte recruitment, immune system activation, tissue fibrosis and tumorigenesis.

The methods comprise administering a therapeutically effective amount of a polypeptide as hereinbefore defined.

### BRIEF DESCRIPTION OF THE DRAWINGS

FIG. 1 comprises alignments of modified dendroaspins useful in the products of the invention where the inserted amino acid sequences (SEQ ID NOS:2 TO 15) are listed beneath the amino acid sequence of dendroaspin (SEQ ID NO: 1).

FIG. 2 shows an HPLC profile of an ND9-mixture.

FIG. 3 shows a diagramatic representation of the structure of ND9.

### EXAMPLE 1

NAP-RGD-Dendroaspin

Restriction enzymes, T4 polynucleotide kinase, T4 DNA ligase, IPTG (isopropyl-β-D-thio-galactopyranoside) and DHSα competent cells were purchased from Life Technologies Ltd (U.K.) or Promega Ltd (Southampton, U.K.). Vent (exo-) DNA polymerase was supplied by New England Biolabs Ltd (Hitchin, U.K.). Proteinase Factor Xa was purchased from Boehringer Mannheim (Sussex, England). Oligonucleotides were made by Cruachem Ltd (Glasgow, U.K.). Deoxynucleotide triphosphates (dNTP's), and plasmid pGEX-3×, a vector that expresses a cloned gene as a fusion protein linked to glutathione S-tranferase (GST), and glutathione-sepharose CL-4B were purchased from Pharmacia Biotech Ltd (Herts, U.K.). Gel extraction kit and Plasmid maxi kit were purchased from Quiegen Ltd (Surrey, U.K.).

Construction of Synthetic Dendroaspin and NAP5 Genes, Respectively

A synthetic dendroaspin gene was constructed by ligating 10 complementary and overlapping oligonucleotides coding for the protein sequence of dendroaspin, using Escherichia coli codon usage data, and cloned into plasmid pGEX-3×. The detailed description of protein expression, purification and functional characterisation of recombinant dendroaspin and comparison with its natural counterpart purified from snake venom has been reported previously [38]. Similarly, the NAP5 gene was constructed by ligating 6 complementary and overlapping oligonudeotides (shown below) coding for the protein sequence of NAP5, using Escherichia coli codon usage data, and cloned into plasmid pGEX-3×. The ligated mixtures were digested with BamH I and EcoR I and then cloned into the restricted vector pGEX-3×.

Overlapping oligonucleotides used to construct NAP5 gene

| | | |
|---|---|---|
| NP1(NM1) | NP3 | RN50 |
| NP2 (NM2) | NP4 | RN60 |

| | |
|---|---|
| The following oligonudeotides were used: | |
| NP1: | |
| dGGG ATC CTT GTC CCG CGT AAA GCT TAC CCG GAA TGC GGT GAA AAC GAA TGG CTT GAC GAC | (SEQ ID NO:16) |
| TGC GGT ACT GAG AAA CCG TGC GAA | |
| NP2: | |
| dGCATTTAGCTTCGCACGGTTTCTGAGTACCGCAGTCGTCAAGCCAT | (SEQ ID NO:17) |
| TCGTTTTCACCGCATTCCGGGTATGCTTTACGCGGGACAAG | |
| NP3: | |
| dGCTAAATGCAACGAAGAACCGCCGGAAGAAGAAGACCCGATCTGCCCGTTCTCGTGGTTGCCTTCTTCCGC | (SEQ ID NO:18) |
| CGGCTTGCGTCTGCAAA | |
| NP4: | |
| dGTAGAAACCGTCTTGCAGACGCAAGCCGGCGGAAGAAGGCAACCACGAGAACGGCAGATCGGGTCTTCT | (SEQ ID NO:19) |
| TCTTCCGGCGGTTCTTCGTT | |
| RN50: | |
| DGACGGTTTCTACCGTGACACTGTCATCGGTGACTGCGTCCGTGAAGAAGAATGCGACCAGCATGAAATCA | (SEQ ID NO:20) |
| TCCATGTCTGAG | |
| RN60: | |
| dAATTCTCAGACATGGATGATTTCATGCTGGTCGCATTCTTCTTCACGGACGCAGTCACCGATGACAGTGTC | (SEQ ID NO:21) |
| ACG | |

Construction of Synthetic ND9

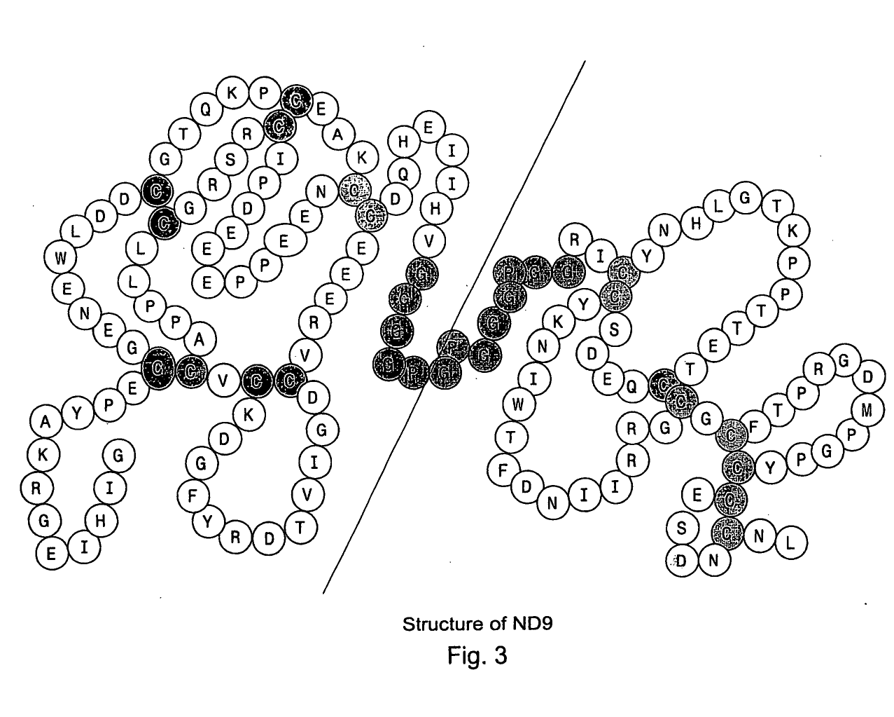

ND9 is a bifunctional molecule which contains two domains, one is a NAP domain and the other a dendroaspin RGD domain. FIG. 3 shows a diagramatic representation of the structure of ND9. The ND9 gene was synthesised from two modified fragments: one is modified NAP5 gene and the other is modified dendroaspin gene. The former was amplified by polymerase chain reaction (PCR), using a mixture of 2 units of vent polymerase, 1 μl of wild-type NAP5 gene (5 ng) as a template, 1 μl of each of two 5′-overhanging deoxyoligonucleotides composed of a forward primer 5′-GGGATCCATATC GMGGT CGTAAAGCTTACCCGGMTGCGGT (xnp5) (SEQ ID NO:22), which contains a sequence for coding IEGR, a non-specific thrombin cleavage site for releasing (by cleavage by thrombin) expressed protein from GST, and a reverse primer 5′-ATGCCCGGGACCACCACCCGGACCACCACCACCGACATGGATGATTTCATGCTG (xnp3) (SEQ ID NO:23), which contains a designed linker and a restriction enzyme (Sma I) site for the linking of the fragment of dendroaspin gene. The latter was amplified also by PCR, using a mixture of 2 units of vent polymerase, 1 μl of wild-type dendroaspin gene (5 ng) as a template, 1 μl of each of two 5′-overhanging deoxyoligonucleotides composed of a forward primer 5′-GGGCCCGGGGGGCGTA TCTGCTACAACCAT (SEQ ID NO:24), which also contains a Sma I site for the linking of the modified NAP5 gene fragment and a reverse primer 5′-AATTCTCAAAGGTGCATTTGTCA GATTCGCAGCAGTACGGACCCGGCATGTCACCACGAGCAGTGAAGCA (SEQ ID NO:25), which substitutes ACG into AGC (GCT into CGT; P into A) for avoiding thrombin cleavage after translation into protein. The modified NAP5 gene was digested with BamH I and Sma I and the modified dendroaspin gene with Sma I and EcoR I, then, ligated with PGEX-3×vector restricted by BamH I and EcoR I to form a complete ND9 gene (SEQ ID NO:26).

Transformation and Protein Expression

Transformation and protein expression were as performed previously [38]. In brief, recombinant plasmid (−5 ng) was used to transform 50 μl of E. coli DH5α competent cells. The presence of the correct coding sequence of the constructs was verified by complete DNA sequencing of the inserted fragments using the dideoxy chain-termination method. Bacterial culture conditions were carried out as follows; the culture was inoculated with an overnight seed culture (1%, v/v) and grown in 2YT/ampillin medium (100 μg/ml) and shaken at 37° C. until it reached an A₆₀₀ of 0.7, then IPTG was added to a final concentration of 0.1 mM for induction. The cells were grown for an additional 4 hours at a low temperature of 30° C. and harvested by centrifugation.

Purification of recombinant ND9

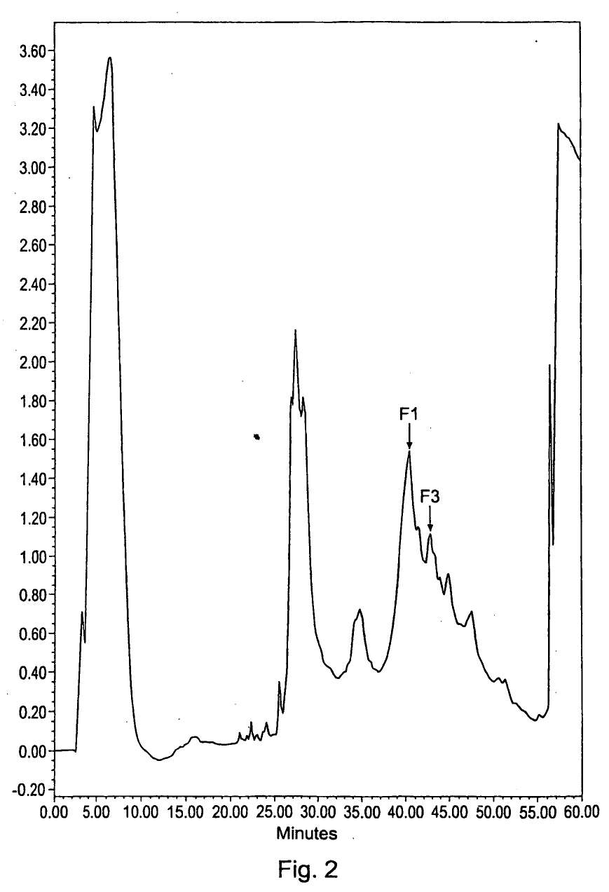

Purification of recombinant ND9 was performed as described previously [38]. In brief, recombinant GST-ND9 from supematants of sonicated cell debris after centrifugation was purified by affinity chromatography on Glutathione-sepharose CLUB columns. The recombinant GST-ND9 was digested with thrombin (1:30, w/w thrombin: fusion protein) at 4° C. for 12 hours. After cleavage, the fractions were loaded onto a Vydac C₁₈ reverse phase HPLC analytical column (TP104) and eluted with a gradient of 0-26% acetonitrile (1.78% per min) containing 0.1% TFA, followed by 26-36% acetonitrile in 0.1% TFA (0.25% per min). When necessary, further analytical columns were run under the same conditions. FIG. 2 shows the elution profile after purification through one column; the F1 and F3 peaks are indicated, it has been found that a large F1 peak can normally be obtained by freezing the sample before running HPLC. The fractions from HPLC were freeze-dried, dissolved in water and assayed for the inhibition of ADP-induced platelet aggregation and Xa inhibition. The results for three fractions are shown below.

Platelet Aggregation Assay

Platelet aggregation was measured by the increase in light transmission as described previously [2]. Briefly, PRP was prepared from citrated human blood, obtained from healthy individuals, by centrifugation at 200×g for 15 mins. Platelet aggregation was induced with 10 μM ADP and measured using a Payton Dual-Aggregometer linked to a chart recorder.

Factor Xa Inhibition Assay

A single-stage chromogenic assay of factor Xa inhibition was used to determine this recombinant molecule. Clotting factor Xa was diluted to 200 μM in 10 mM Hepes (pH 7.5) containing 0.1% bovine serum albumin and 150 mM NaCl (HBSA). Fifty microliters of sample (diluted at vary concentrations into HSBA) were incubated for 30 min at room temperature with 100 μl of factor Xa in a 96-well microtiter plate. Fifty microliters of 1 mM S-2765, a chromogenic substrate for factor Xa, was then added, and the initial rate (mOD per min) of substrate hydrolysis over 5 min was measured at 405/650 nm by using a automated plate reader.

The amino acid sequences of the recombinant molecule designated ND9-F1 (seq id no:29) and NDF9-F3 (SEQ ID NO:30) are shown below:

| | |
|---|---|
| Sequence of ND9-F1 | |
| G I H I E G R K A Y P E C G E N E W L D D C G T Q K P C E | (SEQ ID NO:29) |
| A K C N E E P P E E E D P I C R S R G C L L L¹ P A C V C K | |
| D G F Y R D T V I G D C V R E E E C D Q H E I I H V G G G | |
| G P G P G G G P G G³R I C Y N H L G T K P P T T E T C Q | |
| E D S C Y K N I W T F D N I I R R G C G C F T A R G D M P | |
| G P Y C C E S D N² C N L | |

The amino acid sequence of NAP5 (SEQ ID NO:28) commences with residue 8 (K or lysine) of ND9-F1 (SEQ ID NO:29). Residues 1 to 7 are the remainder of a linker to GST. In an alternative construct, residues 1 to 7 of ND9-F1 are replaced by the classic thrombin cleavage sequence LVPR.

| | |
|---|---|
| Sequence of ND9-F3 | |
| ⊕R S R G C L L L¹ P A C V C K D G F Y R D T V I G D C V R | (SEQ ID NO:30) |
| E E E C D Q H E I I H V G G G G P G P G G G P G G³ R I C | |
| Y N H L G T K P P T T E T C Q E D S C Y K N I W T F D N I | |
| I R R G C G C F T A R G D M P G P Y C C E S D N² C N L | |

Notes
The symbol ⊕denotes a cleavage site.
¹Residue 52 of ND9-F1 and corresponding residue 7 of ND9-F3 is L
(leucine) which is accidentally mutated from the corresponding P
(proline) residue of the published native sequence (39, 40). This
substitution suggests that the residue is not critical for the activity
of NAPS and that the active domain of the protein does not include the
residue.
²The fourth residue from the C-terminus of the ND9 proteins is N
(asparagine) which is accidentally mutated from the K (lysine) residue
of native dendroaspin. This conservative substitution has not destroyed
the activity of the dendroaspin domain.
³The linker, SEQ ID NO:27 (shown underlined) contains 10 glycines and
three prolines. The glycine residue has no side chain which may avoid
unwanted involvement of the side chain with the main chain; proline
sequences like PGP and GPG give necessary turns for an α-helix to
form a favourable conformation which avoids the sequences containing the
two domains (anti-platelet and anti-Xa) interfering with each other (see
example 4).

Inhibition data are shown below:

| | Inhibition | | Inhibition of collagen- | | | |
|---|---|---|---|---|---|---|
| | of ADP-induced | | induced platelet | | | |
| Compound | platelet aggregation | | aggregation | | Anti-Xa | |
| Integrilin* | 0.2 μg/ml | 277 nM | 0.23 μg/ml | 319 nM | — | — |
| Repro* | 3.2 μg/ml | 76 nM | 3.80 μg/ml | 90 nM | — | — |
| Dendroaspin | 1.2 μg/ml | 120-180 nM | 0.35 μg/ml | 52 nM | — | — |
| TAP | — | — | — | — | 0.02-0.07 μg/ml | 2.8-10 nM |
| ND9-F1^{#} | 8.1 μg/ml | 500 nM | 6.30 μg/ml | 388 nM | 0.018 μg/ml | 1.1 nM |
| ND9-F2^{#} | 6.5 μg/ml | 400 nM | 3.60 μg/ml | 225 nM | 1.5 μg/ml | 91.1 nM |
| ND9-M3^{#} | 4.2 μg/ml | 260 nM | 3.90 μg/ml | 240 nM | 2.3 μg/ml | 140.9 nM |
| NAP | — | — | — | 20000 nM | 0.03-0.16 μg/ml | 3-18 nM |

*Assuming the MW of Repro is 42 Kd and Mw of Integrillin is 720d. Repro, also known as 7E3, is a known inhibitor of integrin-mediated binding.
ND9-F1, F2 and F3 are three fraction of HPLC with a C18 column. F2 is, based on functional data, a mixture of F1 and F2. Of these fractions ND9-F1 is preferred.

### EXAMPLE 2

KGD-Dendroaspin

Materials—Restriction enzymes, T4 polynucleotide kinase, T4 DNA ligase, IPTG (isopropyl-β-D-thio-galactopyranoside) and DH5α competent cells were purchased from Life Technologies Ltd (U.K.) or Promega Ltd. (Southampton, U.K.). Vent (exo-) DNA polymerase was supplied by New England Biolabs Ltd. (Hitchin, U. K.). Proteinase Factor Xa was purchased from Boehringer Mannheim (Sussex, England). Human fibrinogen (grade L) was purchased from Kabi (Stockholm, Sweden). Lyophilised snake venoms were obtained from either Latoxan (05150 Rosans, France) or Sigma Chemical Ltd (Dorset, U. K.). Oligonucleotides were made by Cruachem Ltd., (Glasgow, U. K.) and further purified by denaturing PAGE on a 15% acrylamide/B M urea gel. Deoxynucleotide triphosphates (dNTPs), dideoxynucleotide triphosphates (ddNTPs) and plasmid pGEX-3×, a vector that expresses a doned gene as a fusion protein linked to glutathione S-transferase (GST), and Glutathione-Sepharose CL-4B were purchased from Pharmacia Biotech Ltd. (Herts, U. K.). “Geneclean” kit and Plasmid maxi Kit were purchased from Bio 101, La Jolla Calif. U.S.A. and Qiagen Ltd., Surrey, U.K. respectively. The sequencing enzyme (Sequenase 2.0) was obtained from Cambridge Bioscience (Cambridge, U. K.). [³⁵S]dATP[αS] and 125I (15.3 mCi/mg iodine) were supplied by NEN Dupont (Herts, U.K.) and Amersham International Pic (Amersham, Bucks, England), respectively.

Construction of the expression vectors—A dendroaspin gene was constructed from synthetic oligonucleotides, using the same 10 oligonucleotides shown in FIG. 2A of WO 98/42834. Each purified oligonucleotide was phosphorylated at 37° C. for 60 min in the presence of 1 mM ATP and T4 polynucleotide kinase. Each pair of overlapping phosphorylated oligonucleotides was annealed separately on a Perkin-Elmer/Cetus thermal cycler. The following programme was used: 95° C. 5 min, 70° C. 30s then slowly cooling to room temperature. Ligation was performed at 16° C. for 15 hours in a total volume of 50 μl containing approx. 1 nM of each annealed fragment, 50 mM Tris-HCl (pH 7.6), 10 mM MgCl₂, 1 mM ATP and 5% PEG 8000 and 5 units of T4 DNA ligase. After ligation, the dendroaspin gene was amplified by PCR using 1 μl of ligation mixture as template with oligo 1 and 10 as primers and 2 units of Vent DNA polymerase. The following programme was applied: one cycle of 3 min at 94° C. and 1 min at 72° C., followed by 39 cydes of 30s at 94° C., and 2 min at 72° C. The amplification product was checked and found to be of expected size (216 bp) as ascertained on a 2% agarose gel and further purified on a 2% low-melting-point agarose gel. The dendroaspin gene was digested with EcoRI and BamHI and then cloned into the restriction vector pGEX-3×to produce recombinant plasmid pGEXDendroaspin gene. The same protocol is followed in the construction of the non-wild type expression vectors, for example the pGEX-KGD-Dendroaspin gene (see below).

The KGD-dendroaspin gene was produced by using a Transformer™ site-directed mutagenesis kit (Clontech Laboratories Inc, Palo Alto, Calif., USA). A selection oligonucleotide was designed to introduce a novel restriction site (BamHI→ACC65I) into the PGEX-3× vector to allow selection of recombinant from parental constructs by digestion with ACC65I. After annealing, ligation and digestion, the reaction mixture was transformed into E coli mut S cells (Clontech) and subsequent colonies were screened by ACC65I restriction analysis. After two or three rounds of restriction with ACC65I and transformation, more than 90% recombinant clones were identified. In the mutagenesis procedure, there were used the selection primer dGAAGGTCGTGGGTACCATATCGAAGGTCGT (SEQ ID NO:31) and the mutagenesis primer dTGCTTCACTCCGAAAGGTGACATGCCGGGTCCGTAC (SEQ ID NO:32).

Transformation and protein expression—Recombinant gene (S ng) was used to transform 50 μl of E. coli DHSα competent cells by standard methods (34), The presence of correct coding sequence of the constructs was verified by complete DNA sequencing of the inserted fragments using the dideoxy chain-termination method (35). Bacterial culture conditions were carried out as follows: the culture was inoculated with an overnight seed culture (1%, v/v) and grown in LB/ampidllin medium (100 μg/ml) and shaken at 37° C. until it reached an A₆₀₀ of 0.7, then IPTG was added to a final concentration of 0.1 mM for induction. The cells were grown for an additional 4 hours at a lower temperature of 30° C. and harvested by centrifugation.

Purification of native and recombinant snake venom RGD proteins—Elegantin, and dendroaspin were purified using reverse-phase HPLC as described previously (36). Recombinant dendroaspins were purified as follows: the cell pellets were suspended in PBS buffer (pH 7.4) containing 1% Triton X-100 and the protease inhibitors PMSF (1 μM), pepstatin (5 μg/ml), aprotinin (5 μg/ml), trypsin inhibitor (1 μg/ml), 1 mM EDTA, and sonicated on ice. The sonicated mixture was centrifuged at 7,800×g at 4° C. to pellet the cell debris and insoluble material. Recombinant GST-dendroaspin and GST-mutant-dendroaspins from supernatants were purified by affinity chromatography on glutathione-Sepharose CL-4B columns by absorption in PBS containing 150 mM NaCl and elution with 50 mM Tris-HCl containing 10 mM reduced glutathione (pH 8.0). With the remaining insoluble fusion protein in the pellets, solubilisation was achieved in the presence of 8 M urea, by gently shaking at room temperature for 30 min and subsequent renaturation by continual dilution and dialysis at room temperature against Tris-HCl buffer. The refolded fusion protein mixture was subjected to further centrifugation and affinity-purification. The purification was monitored by SDS-PAGE and the appropriate fractions comprising the recombinant GST-Dendroaspin and GST-mutant-dendroaspins were digested in the presence of 150 mM NaCl, 1 mM CaCl₂ and Factor Xa (1:100, w/w Factor Xa:fusion protein) at 4° C. for 24 hours. After cleavage, the fractions were loaded onto a Vydac C₁₈ reverse phase HPLC analytical column (TP104) and eluted with a gradient of 0-26% acetonitrile (1.78% per min) containing 0.1% trifluoroacetic acid (TFA), followed by 26-36% acetonitrile in 0.1% TFA (0.25% per min). When necessary, further analytical columns were run under the same conditions. The fractions from HPLC were freeze-dried, dissolved in water and assayed for the inhibition of ADP-induced platelet aggregation. Purified wild-type dendroaspin and mutants were characterised by 20% SDS-PAGE and electrospray ionisation mass spectrometry.

Measurement of platelet aggregation—Platelet aggregation was measured by the increase in light transmission as described previously (36, 37). Briefly, platelet rich plasma (PRP) was prepared from citrated human blood, obtained from healthy individuals, by centrifugation at 200×g for 15 min. Washed platelets were prepared from PRP and resuspended in adhesion/aggregation buffer (145 mM NaCl, 5 mM KCl, 1 mM MgCl₂, 2 mM CaCl₂, 10 mM glucose, 3.5 mg/ml BSA and 10 mM HEPES, pH 7.35) and adjusted to a count of 3×10/ml. Platelet aggregation (320 μl incubations) was induced with 10 μM ADP in the presence of 1.67 mg/ml fibrinogen and measured using a Payton Dual-Aggregometer, linked to a chart recorder. KGD-dendroaspin was found to show potent inhibition of ADP-induced platelet aggregation.

Measurement of platelet adhesion—Platelet adhesion is measured as described previously (37). Briefly, 96 well plates are coated overnight at 4° C. with either human fibrinogen or fibronectin reconstituted in phosphate buffered saline (PBS) (pH 7.4) at appropriate concentrations (2-10 μg/ml, 100 μl). Platelets are treated with antagonists at appropriate concentrations for 3 min before the addition (90 μl) to the microtitre plates which are pre-loaded with 10 μl of 500 μM ADP (final conc. 50 μM) and the number of adherent platelets is determined by measurement of endogenous acid phosphatase using 130 μl/well of the developing buffer (sodium acetate, pH 5.5, 10 mM p-nitrophenyl phosphate, 0.1% Triton X-100) and read at 410/630 nm on an automated plate reader.

Iodination of Ligands and Ligand Binding Studies—lodination of all proteins used in this study is performed using Enzymobead Radioiodination Reagent (Blorad Laboratories) according to the manufacturer's specifications. The binding of ¹²⁵I-labelled disintegrins, dendroaspin and mutant dendroaspins to washed platelets is performed under equilibrium conditions essentially as described previously (37). Briefly, the incubation mixture is composed of 300 μl of washed platelets (3×10⁸/ml), 10 μl of agonist (1.75 mM ADP giving a final conc. of 50 μM), 10 μl of ¹²⁵I-labelled protein samples, 5-20 μl resuspension buffer and made to a final volume of 350 μl. In antibody inhibition studies, platelet suspensions are treated with antibody for 30 min prior to exposure to ADP and then added to ¹²⁵I-protein samples and the mixture incubated at room temperature for a further 60 min. Incubations are terminated by loading onto a 25% (w/v) sucrose, 1% BSA cushion and centrifugation at 12,000×g for 10 min. Both platelet pellets and supernatants are counted to determine the levels of bound and free ligand. Background binding levels are determined in the presence of a 50-fold excess of cold disintegrin or 10 mM EDTA.

Expression and purification of recombinant wild-type dendroaspin and mutantdendroaspins—The synthetic wild-type and mutated dendroaspin genes were cloned into the expression vector pGEX-3×at the carboxyl terminus of the glutathione S-transferase (GST) gene with a Factor Xa cleavage sequence positioned 5′ of the gene coding for these recombinant proteins. The expression of the GST-fusion protein in E. coli was induced by addition of IPTG to the growth medium, as described under the headings “Construction of the expression vector” and “Transformation and protein expression”. In contrast to non-induced transformants, analysis of IPTG treated cell lysates by SDS-PAGE showed an emergence of a 32 kDa protein corresponding to the GST-fusion protein. The GST-protein was purified by affinity chromatography on glutathlone-Sepharose CL-4B column and monitored by SDS-PAGE. Elution of the absorbed material with glutathione resulted in the appearance of a major band migrating at 32 kDa and a minor band at 28 kDa in 12.5% polyacrylamide gels. This minor 28 kDa component may correspond to free GST released from the GST-protein by an endogenous bacterial protease with Factor Xa-like activity since the relative levels of this species varied with different preparations. Treatment of the purified GST-proteins with Factor Xa released recombinant proteins migrating as 7 kDa bands, approximating the size of dendroaspin, and free GST appearing as an intensification of the 28 kDa band identified by SDS-PAGE. The 7 kDa protein was further purified to homogeneity by reverse-phase HPLC with the active fraction identified by testing aliquots from each peak for their ability to inhibit ADP-induced platelet aggregation in PRP. Further characterisation by mass spectrometry confirmed the successful cleavage at Arg¹ by Factor Xa protease treatment.

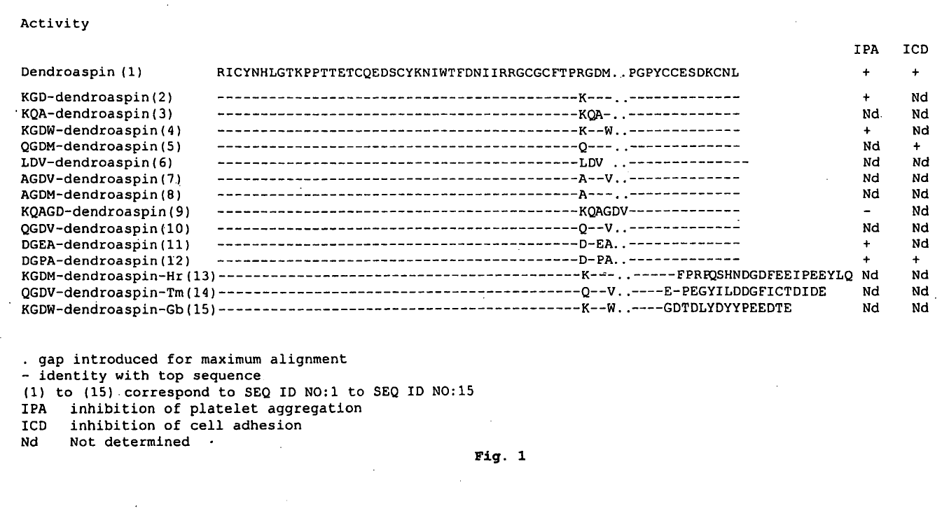

Modified Molecules—FIG. 1 shows the sequences of modified monofunctional and bifunctional dendroaspins obtainable by mutagenesis of the dendroaspin gene as described in the specification and in WO 98/42834. The sequences of these molecules are shown in the sequence listing.

### EXAMPLE 3

KQAGDV-Dendroaspin

The same procedures as described in Example 2 were followed to express and purify KQAGDV-dendroaspin. The mutagenesis primer used in the site-directed mutagenesis was: dGGT TGC TTC ACT CCG AAA CAG GCT GGT GAC GTT CCG GGT CCG TAC TGC (SEQ ID NO:33), corresponding to the amino acid sequence: GCFTPKQAGDVPGPYC (SEQ ID NO:34).

### EXAMPLE 4

In this example the role of proline residues flanking the RGD motif of dendroaspin was investigated. Five dendroaspin variants were prepared with either single or double proline to alanine substitutions namely A⁴²-Den, A⁴⁷-Den, A⁴⁹-Den, A^{42,47}-Den and A^{47,49}-Den.

Inhibition of Platelet Aggregation by Mutants of Dendroaspin

The efficacy of wild-type and five variants of dendroaspin was assessed using ADP-induced platelet aggregation in PRP. The percentage of inhibition of platelet aggregation by these mutant dendroaspins was compared with wild-type dendroaspin and the inhibition of platelet aggregation expressed as IC₅₀ values. A⁴²-Den, A⁴′-Den and A⁴⁹-Den were approx equal potent inhibitors of ADP-induced platelet aggregation in PRP, with IC₅₀ values of ˜170 nM. They showed similar potency to wild-type dendroaspin. In contrast, both mutants with double alanine substitutions, A^{42,47}-Den, and A^{47,49}-Den, were approx. 6-8-fold less potent inhibitors of platelet aggregation than wild-type dendroaspin, with IC₅₀ values of ˜1.3 nM.

Inhibition of A375 (β₃ Integrins) Adhesion to Fibrinogen

The inhibitory efficacy of wild-type and five variants of dendroaspin for A375-SM cell adhesion was also determined in the presence of Mn²+cations. A375-SM cell adhesion on fibrinogen was maximal at a Mn²+concentration of 500 μM.

Under assay conditions, the percentage of inhibition of A375 cell adhesion to fibrinogen by these mutant dendroaspins shows similar patterns to those seen in the platelet aggregation assay. A⁴²-Den, A⁴⁷-Den and A⁴⁹-Den were approx. equally potent inhibitors of A375 cell adhesion to fibrinogen as wild-type dendroaspin at a concentration of 10 nM. In contrast, both mutants with double alanine substitutions, A^{42,47}-Den and A^{47,49}-Den, were approx. 5-fold less potent inhibitors of cell adhesion than wild-type dendroaspin even with as double concentration as that of wild-type dendroaspin (FIG. 3C).

Discussion

Both ADP-induced platelet aggregation and cell adhesion assays for determining the effect of mutagenesis of proline residues on the potency of inhibition of platelet aggregation and. A375-SM cell adhesion. The results showed that a single alanine for proline substitution did not affect potency either of inhibiting platelet aggregation or A375 cell adhesion to fibrinogen, producing similar potency to that of wild-type dendroaspin. By comparison, double proline to alanine substitutions significantly reduced the potency in both assays by approx. 5-8-fold. This suggests that neither proline is essential for binding to the integrin, but loss of both prolines may affect structure of the loop.

The results suggest that the presentation of the proline provides a favourable environment for the RGD-antiplatelet activity. This property may be due to the nature of the proline residue, the only common imino acid in proteins with a bulky pyrrolidine ring that restricts the conformational range of adjacent residues. The lack of a proton on the imino nitrogen blocks hydrogen bond formation required for α-helix and β-sheet secondary structure, and thus disrupts the propagation of neighbouring secondary structures through interactive site(s). Proline also causes kinks and bends in the protein structure that, in dendroaspin, diverge the RGD-loop from the β-sheet [Sutcliffe, M. J., Jaseja, M., Hyde, E. I., Lu, X. and Wiliams, J. A. (1994) Three-dimensional structure of the RGD-containing neurotoxin homologue dendroaspin. Nat. Struct. Biol. 1, 802-807]. Thus proline residues, particularly in either flanking the RGD sequence (P⁴2RGDMP⁴⁷) or neighbouring the RGD sequence (RGDMP⁴⁷GP⁴⁹), provide a favourable conformation for the RGD site in which it is exposed in solvent and recognised by integrin receptor, such as integrin α_{IIb}β₃. It is possible that the middle residue glycine in the P⁴⁷GP⁴⁹ sequence may also contribute to assist in “presentation” of the RGD site due to the lack of side chain

### REFERENCES

1. Kieffer, N., and Phillips, D. R. (1990) Ann. Rev. Cell Biol. 6, 329-357

2. Hynes, R. O. (1992) Cell 69, 11-25

3. McEver, R. P. (1992) Curr. Opin. Cell Biol 4, 840-849

4. Smyth, S. S., Joneckis, C. C., and Parise, L V. (1993) Blood 81, 2827-2843

5. Giancotb, F. G. and Mainiero, F. (1994) Biochim. Siophys. Acta. 1198, 47-64

6. Pierschbacher, M. D., and Ruoslahti, E. (1984) Nature 309, 30-33

7. Plow, E. F., Pierschbacher, M. D., Ruoslahti, E., Marguerie G., Ginsberg, M. H. (1987) Blood 70, 110-115

8. Pytela, R., Pierschbacher, M. D., Ginsberg, M. H., Plow, E. F., Ruoslahti, E. (1986) Science 231, 1559-1162

9. Nachman, R. L., & Nachman, L. L. K. (1992) J. Clin. Invest. 69, 263-269

10. Gardner, J. M. & Hynes, R. O. (1985). Cell 42, 438-449

11. Ruggeri, Z. M., De Mario, L., Gath, L., Bader, R., & Montgomery, R. R. (1983) J. Cin. Invest. 72, 1-12

12. Pytela, R. M., Pierschbacher, M. D. & Rouslahti, E. (1985) Proc. Natl. Acad, Sci USA 82, 5766-5770

13. Karczewski, J. K., Knudsen, A., Smith, L., Murphy, A., Rothman, V. L. & Tuszinski, G. P. (1989) J. Biol Chem. 264, 21322-21326

14. Sims, P. J., Ginsberg, M. H., Plow, E. F. and Shattil, S. J. (1991) J. Biol. Chem. 266, 7345-7352

15. Ugarova, T., Agbanyo, F. R., and Plow E. F. Thrombosis and Haemostasis (1995) 74, 253-257

16. Gould, R. J., Polokoff, M. A., Friedman, P. A., Huang, T. F., Holt, J. C., Cook, J. J. and Niewiarowski, S. (1990) Proc Soc. Exp. Bio. Med. 195, 168-171

17. Scarborough, R. M., Rose, J. W., Hsu, M. A., Phillips, D. R., Fried, V. A., Campbell, A. M., Nannizzi, L. and Charo, I. F. (1991) J. Biol. Chem. 266, 9359-9362

18. McDowell, R. S., Dennis, M. S., Louie, A. Shuster, M., Mulkerrin, M. G. and Lasarus, R. A. (1992) Biochemistry 31, 4766-4772

19. Williams J. A., Lu, X., Rahman, S., Keating, C. & Kakkar V. V. (1992) Biochem. Soc. Trans. 21, 73S

20. Knapp, A., Degenhardt, T. & Dod, J. (1992) J. Biol. Chem. 267, 24230-24234

21. Adler, M., Lazarus, R. A., Dennis, M. S. & Wagner, G. (1991) Science 253, 445448

22. Adler, M. & Wagner, G (1992) Biochemistry31, 1031-1039

23. Adler, M., Carter, P., Lazarus, R. A. & Wagner, G. (1993) Biochemistry 32, 282-289

24. Senn, H. & Klaus, W. (1993) J. Mol. Biol. 234, 907-925

25. Saudek, V., Atkinson, R. A. & Pelton, J. T. (1991a) Biochemistry 30, 7369-7372

26. Saudek, V., Atkinson, R. A., Lepage, P. & Pelton, J. T. (1991b) Eur. J. Biochem. 202, 329-338

27. Cooke, R. M., Carter, B. G., Martin, D. M. A., Murray-Rust, P. & Weir, M. P. (1991) Eur. J. Biochem. 202, 323-328

28. Cooke, R. M., Carter, B. G., Murray-Rust, P., Hartshorn, M. J., Herzyk, P. & Hubbard, R. E. (1992) Protein Eng. 5, 473-477

29. Jaseja, M., Smith, K. I., Lu, X., Williams, J. A., Trayer, H., Trayer, I. P., Hyde, E. I. (1993) Eur. J. Biochem. 218, 853-860

30. Krezel, A. M., Wagner, G., Seymour-Ulmer, J. & Lazarus, R. A. (1994) Science 264, 1944-1947

31. Jaseja, M., Lu, X., Williams, J. A., Sutcliffe, M. J., Kakkar, V. V., Parslow, R. A., and Hyde, E. I. (1994) Eur. J. Biochem. 226, 861-868

32. Sutcliffe, M. J., Jaseja, M., Hyde, E. I., Lu, X., Williams, J. A. (1994) Nature Structure Biology 1, 802-807

33. Homby, E. J., Brown, S., Wilkinson, J. M., Mattock, C. & Authi, K. S. (1991) Br. J. Haematol. 79, 277-285

34. Sambrook, J., Fritsch, E. F. & Maniatis, T. (1989) Molecular cloning: A Laboratory Manual, Cold Spring Harbor Laboratory Press, Cold Spring Harbor, N.Y.

35. Sanger, F. S., Nicklen, S. & Coulson, A. R. (1977) Proc. Natl. Acad. Sci. U.S.A 74, 5463-5467

36. Lu, X., Deadman, J. J., Williams, J. A., Kakkar, V. V. & Rahman, S. (1993) Biochem. J. 296, 21-24

37. Lu, X., Williams, J. A., Salmon, G. P., Kakkar, V., Wilkinson, J. M., Baruch, D., Authi, K. S. & Rahmna, S. (1994) Biochem. J. 304, 929-936

38. Lu X, Rahman, S, Kakkar, V. V. and Authi, K. S. (1996) J. Biol. Chem. 271, 289-294

39. Stanssens, P. Bergum, P. W., Ganseman, Y., lespers, L., Laroche, Y., Huang, S., Maki, S., Messens, J., Lauwereys, M., Cappello, M., Hotez, P. J., Lasters, I. and Vlasuk, G. P. (1996) Proc. Natl. Acad. Sci. USA 93, 2149-2154.

40. Duggan, B. M., Dyson, H. J., and Wright, P. E. (1999) Eur. J. Biochem. 265, 539-548.

41. Rebello, S. S., Blank, H. S., Rote, W. E., Vlasuk, G. P. and Lucchesi, B. R. (1997) J. Pharmacol Exp. Therapeutics 285, 91-99.

42. Waxman L., Smith D. E., Arcuri K. E., Vlasuk G. P., (1990) Science, 248(4955):593-6

1. A product comprising
a dendroaspin scaffold, optionally in which the native RGD motif has been deleted or has been replaced by a replacement amino acid sequence, which optionally is (i) an amino acid sequence having no integrin-binding activity or (ii) an integrin-binding amino acid sequence and comprising a tripeptide sequence other than RGD containing D or E adjacent to G, and
a second portion comprising a serine protease inhibitor domain ligated to the dendroaspin scaffold.

2. A product of claim 1 wherein the second portion is ligated to the N-terminus of the dendroaspin scaffold.

3. A product comprising a first portion which is an integrin-binding protein, a homologue thereof having a binding activity, or a fragment of either which has a binding activity, and, ligated to the first portion, a second portion which has a different function.

4. A product of claim 3 wherein said different function is a serine protease inhibitor function.

5. A product of claim 3 or claim 4 wherein the homologue has at least 50% amino acid sequence homology with the wild-type protein.

6. A product of claim 5 wherein the homologue has at least 65% amino acid sequence homology with the wild-type protein.

7. A product of claim 6 wherein the homologue has at least 75% amino acid sequence homology with the wild-type protein.

8. A product of claim 7 wherein the homologue has at least 85% amino acid sequence homology with the wild-type protein.

9. A product of any of claims 3 to 8 wherein the wild-type protein comprises an integrin-binding sequence which is RGD or KGD.

10. A product of claim 9 wherein the homologue contains in place of its native integrin-binding sequence another integrin-binding sequence comprising a tripeptide sequence containing D or E adjacent to G.

11. A product of any of claims 1 to 10 wherein the second portion comprises a protein or a polypeptide.

12. A product of claim 11 wherein the protein or polypeptide of the second portion has a wild-type sequence.

13. A product of claim 11 wherein the protein or polypeptide of the second portion has a sequence which is a modification of a wild-type sequence.

14. A product of claim 13 wherein the protein or polypeptide has a degree of homology with its wild-type protein which is at least 50%, preferably at least 65%, more preferably at least 75% and most preferably at least 85%.

15. A product of any of claims 11 to 14 wherein the protein or polypeptide of the second portion comprises a TAP protein, a NAP protein or an ACAP protein or a homologue thereof having factor Xa inhibitor function.

16. A product of claim 15 wherein the protein or polypeptide comprises NAP5 or a homologue thereof having factor Xa inhibitor function.

17. A product of any of claims 1 to 10 wherein the second portion comprises a fragment of a protein or polypeptide referred to in claim 15 or claim 16.

18. A product of any of claims 1 to 17 wherein the ligation is through a linker.

19. A product of claim 18 wherein the linker comprises a poly(amino acid) which comprises at least one imino acid residue.

20. A product of claim 19 wherein the poly(amino acid) has from 5 to 20 amino acid residues.

21. A product of claim 19 or claim 20 wherein the linker comprises at least a pair of non-adjacent imino acidresidues.

22. A product of any of claims 19 to 21 wherein the linker contains from 2 to 5 imino acidresidues.

23. A product of any of claims 19 to 22 wherein the or each imino acidresidue is adjacent a glycine.

24. A product of claim 23 wherein the linker comprises a region of at least 5 amino add residues consisting solely of glycine and imino acidresidues.

25. A product of claim 24 wherein the number of amino acid residues in said region is from 5 to 20.

26. A product of any of claims 21 to 25 wherein the imino acid is proline.

27. A product of claim 26 wherein the linker comprises a PGP sequence.

28. A product of claim 24 or claim 25 wherein the region comprises the sequence GPGP(G)ₙPG, where n is from 1 to 10.

29. A product of any of claims 11 to 16, or 17 to 28 when dependent (directly or indirectly) on claim 11, wherein said protein is a snake venom protein and optionally is a disintegrin, applaggin, kistrin, echstatin, flavoridin, albolabrin, decorsin or dendroaspin.

30. A product of claim 1 or claim 10 in which the tripeptide sequence is of the formula
B-J-Z
wherein
I) J-Z is GD or GE and B is R, K, Q, A, H, N, A, V, I, L, M, F, P or W;
II) B-J is DG or EG and Z is any amino acid; or
III) J is D or E and B and Z are each independently selected from A, V, I, L, M, F, P or W.

31. A products of claim 30(I) in which J-Z is GD.

32. A products of claim 30(I) or claim 31 in which B is R, K, Q, A, H or N.

33. A product of claim 31 in which B is R, K, Q or A.

34. A product of any of claims 30(I) and 31 to 33 in which B-J-Z is included at its C-terminal end to M, W, N or V.

35. A product of claim 34 in which said M, W, N or V residue is followed by the P which is at position 47 of wild type dendroaspin or by an A residue substituted therefor.

36. A product of any of claims 30(I) and 31 to 35 in which the snake venom protein of claim 2 is dendroaspin and the integrin-binding amino acid sequence is preceded by the P which is at position 42 of wild type dendroaspin or by an A residue substituted therefor.

37. A product of claim 30(I) or 31 in which B is A, V, I, M, F, P, W.

38. A product of claim 37 in which B is L or V.

39. A product of claim 35 in which B is L and is preceded by M.

40. A product of claim 30(II) in which B-J is DG.

41. A product of claim 30(II) or claim 40 in which Z is E, R or P.

42. A product of any of claims 30(II), 40 and 41 in which the snake venom protein of claim 3 is dendroaspin and Z is followed by the P which is at position 47 of wild type dendroaspin or by an A inserted before the wild type position 47 P.

43. A product of claim 40 (III) in which 3 is D.

44. A product of claim 40(III) in which B-J-Z is LDV.

45. A product of any of claims 40(III), 43 and 44 in which B-J-Z is preceded by an I residue.

46. A product of claim 1 or claim 2, which comprises a said replacement amino acid sequence (i) having no integrin binding activity, the replacement amino acid sequence having a receptor-binding function; or a product of claim 3 in which the binding activity is receptor-binding activity.

47. A product of claim 1 or claim 2, which comprises a said replacement amino acid sequence (i) having no integrin binding activity, the replacement amino acid sequence being one which in its native polypeptide enters a pocket to function; or a product of claim 3 which comprises a sequence having the binding activity which in its native polypeptide enters a pocket to function.

48. A product of claim 1 or claim 2, or a product of claim 3 in which the snake venom protein is dendroaspin, wherein in either case the amino acids flanking RGD or said integrin binding sequence are modified as compared with wild-type dendroaspin.

49. A product of claim 1 or claim 2, which contains not more than 100 amino acid residues more than native dendroaspin; or a product of claim 3, which contains not more than 100 amino acid residues more than the native snake venom protein.

50. A product of claim 1 wherein the dendroaspin scaffold has GP IIb/IIIa binding function and the second portion is a TAP protein, a NAP protein or an ACAP protein, a homologue thereof or a fragment of the aforesaid, and has Factor Xa inhibitor function; the dendroaspin scaffold being ligated at its N-terminus to the second portion through a linker comprising a region consisting solely of glycine and proline residues.

51. A product of claim 50 wherein the second portion comprises a NAP protein; a homologue of a NAP protein or a fragment of either and has Factor Xa inhibitor function.

52. A product of claim 51 wherein the second portion comprises an amino acid sequence comprised in the sequence of amino acid residues from residue 1 to residue 40 of SEQ ID NO: 30 (ND9-F3) or a homologue thereof.

53. A product of claim 51 wherein the second portion comprises an amino acid sequence comprised in the sequence of amino acid residues from residue 8 to 84 of SEQ ID NO:29 (ND9F1).

54. A product of any of claims 50 to 53 wherein the dendroaspin scaffold has an RGD motif in loop III.

55. A product of claim 54 wherein the non-RGD residues of loop III are unmodified as compared with native dendroaspin or are modified by substitution, insertion and/or deletion of 1, 2 or 3 amino acid residues.

56. A product of claim 55 wherein the non-RGD residues are unmodified or are modified by substitution of 1, 2 or 3 amino acid residues.

57. A product of claim 56 wherein loop III has the sequence of loop III of ND9-F1(residues 93 to 103 of SEQ ID NO: 29.

58. A product of claim 56 wherein loop III has the sequence of loop III of native dendroaspin (residues 40 to 50 of SEQ ID NO:1).

59. A product of any of claims 53 to 58 wherein the dendroaspin scaffold outside loop III is unmodified as compared with dendroaspin or is modified by substitution, insertion and/or deletion of from 1 to 10 residues.

60. A product of claim 59 wherein the dendroaspin scaffold outside loop m is unmodified or is modified by substitution, insertion and/or deletion of 1, 2 or 3 residues.

61. A product of any of claims 50 to 53 wherein the dendroaspin scaffold has the sequence of native dendroaspin (SEQ ID NO:1) or the sequence of residues 54 to 112 of SEQ ID NO:30 (ND9F3).

62. A product of any of claims 50 to 61 wherein the linker is as defined in any of claims 19 to 28.

63. A poly(amino acid) comprising a NAP-based domain having serine protease inhibitor activity linked through a proline-containing domain to another domain having integrin binding activity.

64. A poly(amino acid) of claim 63 wherein said other domain comprises a dendroaspin sequence.

65. A hybrid poly(amino acid) comprising two domains, not both derived from the same native molecule, interlinked by a linker comprising an imino acid residue.

66. A poly(amino acid) of claim 65 wherein the linker comprises at least two non-adjacent imino acid residues.

67. A poly(amino acid) of claim 66 wherein the linker contains from 2 to 5 imino acid residues.

68. A poly(amino acid) of claim 67 wherein the non-adjacent imino acid residues comprise a pair of imino acid residues separated by from 1 to 10 amino acid residues.

69. A poly(amino acid) of any of claims 65 to 68 wherein at least one imino acid is adjacent a glycine residue.

70. A poly(amino acid) of any of claims 66 to 68 wherein the linker comprises at least two non-adjacent imino acid residues which are adjacent a glycine residue.

71. A poly(amino acid) of claim 66 wherein the linker comprises a sequence IA-(G)ₙ-IA, where each IA independently is an imino acid, G is glycine and n is from 1 to 10.

72. A poly(amino acid) of claim 71 wherein the linker comprises the sequence IA-G-IA.

73. A poly(amino add) of claim 71 wherein the linker comprises the sequence G-IA-G-IA-G₍ₙ₎-IA-G.

74. A poly(amino acid) of any of claims 65 to 73 wherein each imino acid is proline.

75. A poly(amino acid) of any of claims 65 to 74 wherein one of the two domains has platelet-binding activity and the other of the two domains has an activity selected from the group consisting of platelet binding activity, anticoagulant activity, antithrombotic activity, inhibition of cell migration, inhibition of cell proliferation, inhibition of a component in the dotting cascade and regulation of signal transduction.

76. A poly(amino acid) of any of claims 65 to 75 wherein one of the two domains confers GPIIb/IIIa binding activity and the other of the two domains confers platelet derived growth factor (PDGF) activity, glycoprotein IBα activity, hirudin activity, thrombomodulin activity, vascular epidermal growth factor activity, transforming growth factor-β1 activity, basic fibroblast growth factor activity, angiotensin II activity, factor VIII activity, von Willebrand factor activity, tick anticoagulant protein (TAP) activity or nematode anticoagulant protein (NAP) activity.

77. A poly(amino acid) of claim 76, wherein the other of the two domains comprises a sequence derived from platelet derived growth factor (PDGF), glycoprotein IBα, hirudin, thrombomodulin, vascular epidermal growth factor, transforming growth factor-β1, basic fibroblast growth factor, angiotensin II, factor VIII, von Willebrand factor, tick anticoagulant protein (TAP) or nematode anticoagulant protein (NAP), or a sequence having homology to at least part of such sequence.

78. A nucleic acid molecule encoding a polypeptide product of any one of claims 1 to 77.

79. A nucleic acid of claim 78, operatively linked to a promoter and optionally to a nucleic acid sequence encoding a heterologous protein or peptide thereby to encode a fusion product.

80. A nucleic add of claim 79, wherein the promoter is IPTG inducible and optionally the heterologous protein or peptide is glutathione S-transferase.

81. A plasmid comprising a nucleic acid of any one of claims 78 to 80.

82. Plasmid pGEX-3×comprising a nucleic acid of claim 78.

83. A host cell transformed with a plasmid of claim 81 or claim 82.

84. A host cell of claim 83 which is E coli.

85. A cell culture comprising host cells of claim 83 or claim 84.

86. A method of producing a polypeptide product of claims 1 to 77 comprising culturing a host cell of claim 83 or claim 84 so as to express said polypeptide, extracting the polypeptide from the culture and purifying it.

87. A method of producing a polypeptide comprising an integrin-binding protein or its homologue, the method comprising:
a) preparing an expression vector comprising a nucleic add sequence encoding a polypeptide product of claim 3 operatively linked to a promoter and optionally linked to a nucleic acid sequence encoding a heterologous affinity purification protein for co-expression therewith; and
b) transforming a host cell with the vector and causing the host cell to express the nucleic acid sequence.

88. A method of claim 87, wherein step (a) comprises
(i) assembling from overlapping oligonucleotides the coding sequence of an integr-inbinding protein or a homologue thereof having a binding activity;
(ii) assembling from overlapping oligonucleotides the coding sequence of the second portion;
(iii) amplifying the coding sequences, the PCR primers being designed to allow cloning of the integrin-binding protein and the second portion into an expression vector, the PCR primers optionally encoding a linker to interlink the integrin-binding protein and the second portion;
(iv) preparing an expression vector comprising the coding sequences operatively linked to a promoter and optionally linked to a nucleic acid sequence encoding a heterologous affinity purification protein for co-expression therewith.

89. A method of claim 88, which further comprises modifying the nucleic acid sequence of the vector by one or more of the insertion, deletion or substitution of nudeic acid residues.

90. A method of claim 87, wherein step (a) comprises constructing from oligonucleotides an expression vector comprising a nucleic acid sequence encoding a dendroaspin sequence in which the RGD-encoding domain has been deleted or replaced by a replacement amino acid sequence as defined in any of claims 30 to 45 and, optionally, modifying at least one other domain of the nucleic acid sequence of the vector encoding the dendroaspin scaffold by one or more of insertion, deletion or substitution of nucleic acid residues so that on expression the dendroaspin scaffold comprises a corresponding domain having a non-wild-type dendroaspin sequence.

91. A method of any of claims 86 to 90, further comprising the steps of:
d) extracting the expressed polypeptide from a host cell culture,
e) purifying the polypeptide from the cell culture extract, and, if the polypeptide is a fusion protein with a heterologous affinity purification protein, cleaving the desired product from the heterologous affinity purification portion of the fusion protein.

92. A method of claim 91 wherein the heterologous affinity purification protein is glutathione S-transferase (GST) and the purification involves GST affinity chromatography followed by cleavage of the modified dendroaspin from GST.

93. A polypeptide product of any of claims 1 to 77 obtainable by the method of any one of claims 86 to 92.

94. A pharmaceutical composition comprising a pharmacologically active product of any one of claims 1 to 77 or 93.

95. A composition as claimed in claim 94, further comprising a pharmaceutically acceptable excipient or carrier.

96. A pharmacologically active product as claimed in any one of claims 1 to 77 or 93 for use as a pharmaceutical.

97. The use of a pharmacologically active product as claimed in any one of claims 1 to 77 or 93 for the manufacture of a medicament for the treatment or prophylaxis of disease associated with thrombosis.

98. The use as claimed in claim 97, wherein said disease is one of more of thrombosis, myocardial infarction, retinal neovascularization and endothelial injury.

99. A method for the treatment or prophylaxis of a disease associated with thrombosis in a human or animal patient, comprising administering to the patient an effective amount of a pharmacologically active product as claimed in any one of claims 1 to 66 or 77.

100. A linker comprising an amino acid sequence selected from the group consisting of Aa1-Gly and Gly-Aa1, wherein Aa1 is an imino add.

101. A linker of claim 100 wherein said sequence is contained in the amino acid sequence Aa1-Gly-Aa2, where Aa1 and Aa2 are each independently imino acids.

102. A linker of claim 101 wherein said sequence is contained in the amino add sequence Gly-Aa1-Gly-Aa2-Gly.

103. A linker any of claims 100 to 102 wherein Aa1 and Aa2 are both proline.

104. A linker which comprises at least two non-adjacent imino acids.

105. A linker of any of claims 100 to 104 which consists of a poly(amino acid) sequence containing from 5 to 20 amino acids.

106. A product comprising first and second biologically active moieties linked through a linker of any of claims 100 to 105.

107. A product of claim 106 wherein at least one of said moieties is a protein.

108. A product of claim 107 wherein both of said moieties are proteins.
